(19) United States
(12) Patent Application Publication
(10) Pub. No.: US 2025/0076758 A1
(43) Pub. Date: Mar. 6, 2025

(54) PHOTO ACID GENERATOR

(71) Applicant: TAIWAN SEMICONDUCTOR MANUFACTURING COMPANY, LTD., Hsinchu (TW)

(72) Inventors: Chien-Chih CHEN, Taipei City (TW); Ching-Yu CHANG, Yilang County (TW)

(73) Assignee: TAIWAN SEMICONDUCTOR MANUFACTURING COMPANY, LTD., Hsinchu (TW)

(21) Appl. No.: 18/459,077

(22) Filed: Aug. 31, 2023

(51) Int. Cl. G03F 7/004

(52) U.S. Cl. CPC G03F 7/0045

(57) ABSTRACT

A photo acid generator includes a photoactive cation and an anion. The photoactive cation includes a moiety and one or more EUV absorbing atoms. The moiety includes onium salts, selenium salts, phosphonium salts, iodonium salts, or sulfonium salts. The one or more EUV absorbing atoms are attached to the moiety. The anion is attached to the photoactive cation.

### BACKGROUND

As consumer devices have gotten smaller and smaller in response to consumer demand, the individual components of these devices have necessarily decreased in size as well. Semiconductor devices, which make up a major component of devices such as mobile phones, computer tablets, and the like, have been pressured to become smaller and smaller, with a corresponding pressure on the individual devices (e.g., transistors, resistors, capacitors, etc.) within the semiconductor devices to also be reduced in size.

One enabling technology that is used in the manufacturing processes of semiconductor devices is the use of photolithographic materials. Such materials are applied to a surface of a layer to be patterned and then exposed to an energy that has itself been patterned. Such an exposure modifies the chemical and physical properties of the exposed regions of the photosensitive material. This modification, along with the lack of reducing an Extreme ultraviolet (EUV) dose to develop a pattern in the photoresist and reducing a line width roughness (LWR) of the pattern on in regions of the photosensitive material that were not exposed, can be exploited to remove one region without removing the other.

However, as the size of individual devices has decreased, process windows for photolithographic processing has become tighter and tighter. As such, advances in the field of photolithographic processing are necessary to maintain the ability to scale down the devices, and further improvements are needed in order to meet the desired design criteria such that the march towards smaller and smaller components may be maintained.

As the semiconductor industry has progressed into nanometer technology process nodes in pursuit of higher device density, higher performance, and lower costs, there have been challenges in reducing semiconductor feature size. Extreme ultraviolet lithography (EUVL) has been developed to form smaller semiconductor device feature size and increase device density on a semiconductor wafer.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

The present disclosure is best understood from the following detailed description when read with the accompanying figures. It is emphasized that, in accordance with the standard practice in the industry, various features are not drawn to scale and are used for illustration purposes only. In fact, the dimensions of the various features may be arbitrarily increased or reduced for clarity of discussion.

FIG. 1A is a schematic view of an EUV lithography tool with an LPP-based EUV radiation source, in accordance with some embodiments of the present disclosure.

FIG. 1B is a simplified schematic diagram of a detail of an extreme ultraviolet lithography tool according to an embodiment of the disclosure showing the exposure of photoresist coated substrate with a patterned beam of EUV light.

FIG. 2 is a sectional view of a EUV mask constructed in accordance with some embodiments of the present disclosure.

FIG. 3 is a diagrammatic fragmentary cross-sectional side view of a semiconductor device at various stages of fabrication in accordance with various aspects of the present disclosure.

FIG. 4 is a schematic diagram of a photoactive cation of the PAG in accordance with some embodiments.

FIG. 5 is an example structural formula (A1) of the photoactive cation of the PAG in accordance with some embodiments.

FIG. 6 is a diagrammatic fragmentary cross-sectional side view of a semiconductor device at various stages of fabrication in accordance with various aspects of the present disclosure.

FIG. 7A shows an electron generation of a polymer of the photoresist composition upon an EUV radiation in accordance with some embodiments.

FIG. 7B shows formation of an acid “precursor” of the polymer of the photoresist composition in accordance with some embodiments.

FIG. 8A shows an intermolecular charge transfer mechanism of the PAG in accordance with some embodiments.

FIG. 8B shows an intramolecular charge transfer mechanism of the PAG in accordance with some embodiments.

FIG. 8C shows an acid release in accordance with some embodiments.

FIG. 9A shows a graph of EUV absorbing of CH₃—I group of the PAG in accordance with some embodiments.

FIG. 9B is a schematic diagram of highest occupied molecular orbital (HOMO) and lowest unoccupied molecular orbital (LUMO) under UV absorption of the PAG in accordance with some embodiments.

FIGS. 10A and 10B are diagrams of a first mechanism and a second mechanism of the intramolecular charge transfer between donor and acceptor by EUV, respectively, in accordance with some embodiments.

FIGS. 11, 12A, 12B, 13A and 13B are diagrammatic fragmentary cross-sectional side views of a semiconductor device at various stages of fabrication in accordance with various aspects of the present disclosure.

FIGS. 14-19 are diagrammatic fragmentary cross-sectional side views of a semiconductor device at various stages of fabrication in accordance with various aspects of the present disclosure.

FIGS. 20, 21, and 22A illustrate perspective views of additional fabrication processes in the formation of a semiconductor device on a substrate in accordance with some embodiments of the present disclosure.

FIGS. 22B, 23, 24 and 25 illustrate cross-sectional views of additional fabrication processes in the formation of a semiconductor device using a substrate in accordance with some embodiments of the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

As used herein, “around,” “about,” “approximately,” or “substantially” may mean within 20 percent, or within 10 percent, or within 5 percent of a given value or range. One skilled in the art will realize, however, that the value or range recited throughout the description are merely examples, and may be reduced with the down-scaling of the integrated circuits. Numerical quantities given herein are approximate, meaning that the term “around,” “about,” “approximately,” or “substantially” can be inferred if not expressly stated.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

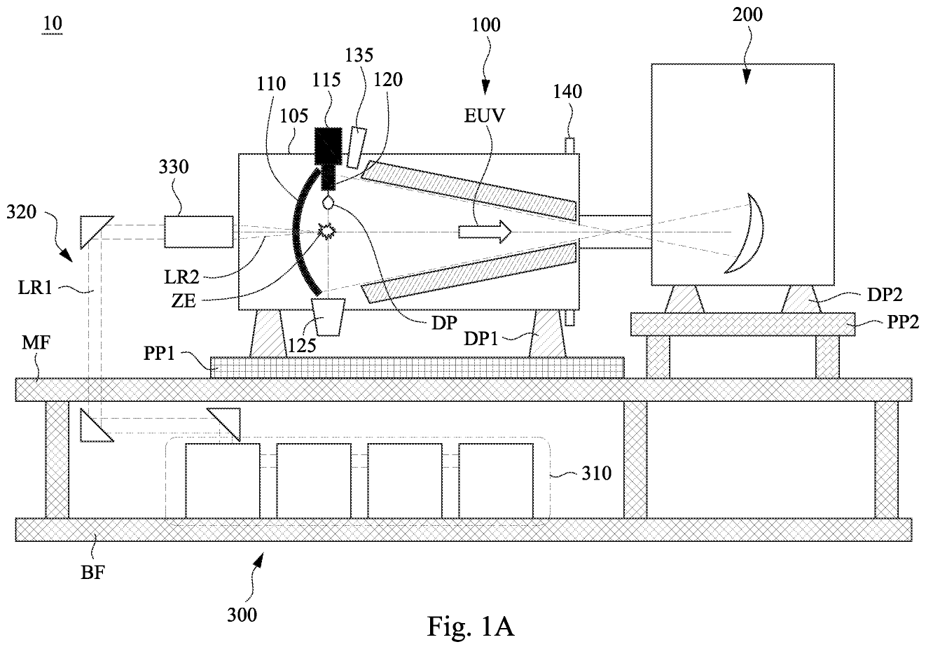

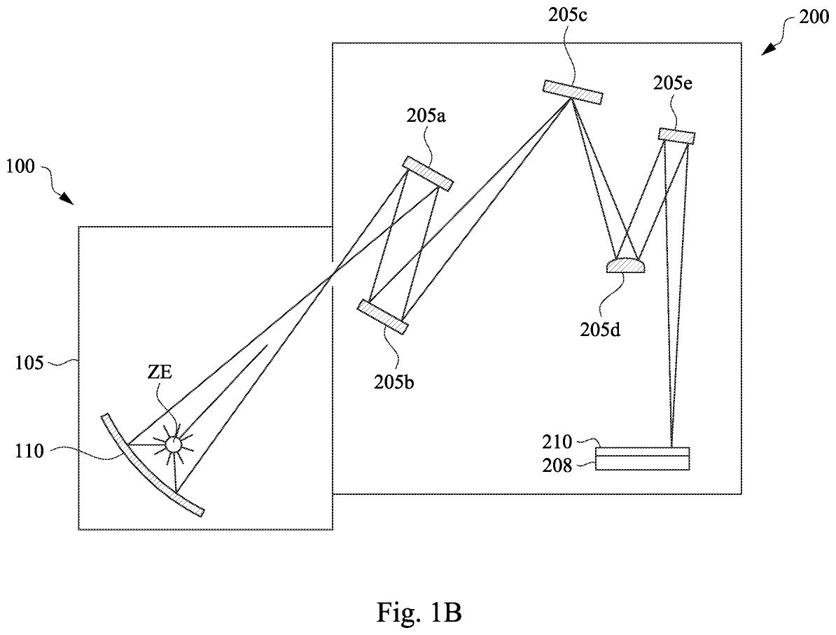

FIG. 1A is a schematic view diagram of an EUV lithography system 10, constructed in accordance with some embodiments. The EUV lithography system 10 may also be generically referred to as a scanner that is configured to perform lithography exposure processes with respective radiation source and exposure mode. The EUV lithography system 10 is designed to expose a photoresist layer by EUV light or EUV radiation. The photoresist layer is a material sensitive to the EUV light. The EUV lithography system 10 employs a radiation source 100 to generate EUV light, such as EUV light having a wavelength ranging between about 1 nm and about 100 nm. In one particular example, the radiation source 100 generates an EUV light with a wavelength centered at about 13.5 nm. Accordingly, the radiation source 100 is also referred to as EUV radiation source 100. Extreme ultraviolet (EUV) lithography has become widely used due to its ability to achieve small semiconductor device sizes, for example for 20 nanometer (nm) technology nodes.

The present disclosure provides a novel photo acid generator (PAG). The PAG may include a high EUV absorbing group and a donor-acceptor design in the same molecule. The PAG has multiple charge transfer mechanisms. In addition to intermolecular charge transfer of the PAG, the high EUV absorbing group can absorb an EUV photon due to its inner-shell electrons and have a rapid intramolecular charge transfer from donor to acceptor. Therefore, a decomposition of the PAG can happen easily and quickly via intramolecular charge transfer. Since the PAG includes both of the intermolecular charge transfer and the intramolecular charge transfer, an acid generation efficiency is improved, which is beneficial for improving lithography performance.

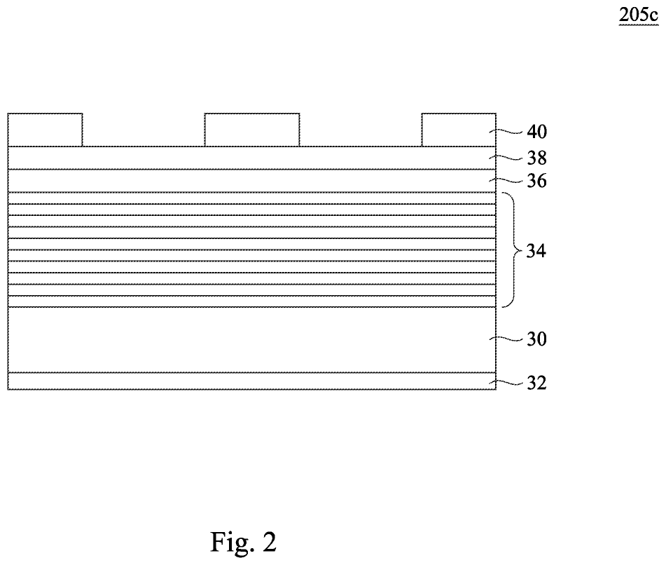

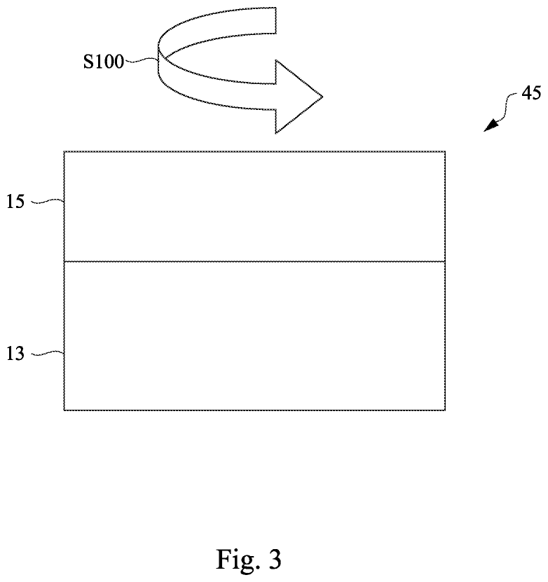

The various aspects of the present disclosure will be discussed below in greater detail with reference to FIGS. 1A-13B. First, an EUV lithography system will be discussed below with reference to FIGS. 1A, 1B and 2. Next, details of the novel PAG and the lithography process employing a photoresist including the novel PAG will be discussed with reference to FIGS. 3-13B.

The advanced lithography process, method, and materials described in the current disclosure can be used in many applications, including fin-type field effect transistors (FinFETs), gate-all-around (GAA) FETs. For example, the fins may be patterned to produce a relatively close spacing between features, for which the above disclosure is well suited. In addition, spacers used in forming fins of FinFETs can be processed according to the above disclosure.

To address the trend of the Moore's law for decreasing size of chip components and the demand of higher computing power chips for mobile electronic devices such as smart phones with computer functions, multi-tasking capabilities, or even with workstation power. Smaller wavelength photolithography exposure systems are desirable. Extreme ultraviolet (EUV) photolithography technique uses an EUV radiation source to emit an EUV light ray with wavelength of about 13.5 nm. Because this wavelength is also in the x-ray radiation wavelength region, the EUV radiation source is also called a soft x-ray radiation source. The EUV light rays emitted from a laser-produced plasma (LPP) are collected by a collector mirror and reflected toward a patterned mask.

FIG. 1A is a schematic view of an EUV lithography tool with an LPP-based EUV radiation source, in accordance with some embodiments of the present disclosure. The EUV lithography system includes an EUV radiation source 100 to generate EUV radiation, an exposure device 200, such as a scanner, and an excitation laser source 300. As shown in FIG. 1A, in some embodiments, the EUV radiation source 100 and the exposure device 200 are installed on a main floor MF of a clean room, while the excitation laser source 300 is installed in a base floor BF located under the main floor MF. Each of the EUV radiation source 100 and the exposure device 200 are placed over pedestal plates PP1 and PP2 via dampers DP1 and DP2, respectively. The EUV radiation source 100 and the exposure device 200 are coupled to each other by a coupling mechanism, which may include a focusing unit.

The EUV lithography tool is designed to expose a resist layer to EUV light (also interchangeably referred to herein as EUV radiation). The resist layer is a material sensitive to the EUV light. The EUV lithography system employs the EUV radiation source 100 to generate EUV light, such as EUV light having a wavelength ranging between about 1 nm and about 100 nm. In one particular example, the EUV radiation source 100 generates an EUV light with a wavelength centered at about 13.5 nm. In the present embodiment, the EUV radiation source 100 utilizes a mechanism of laser-produced plasma (LPP) to generate the EUV radiation.

The exposure device 200 includes various reflective optic components, such as convex/concave/flat mirrors, a mask holding mechanism including a mask stage, and wafer holding mechanism. The EUV radiation EUV generated by the EUV radiation source 100 is guided by the reflective optical components onto a mask secured on the mask stage. In some embodiments, the mask stage includes an electrostatic chuck (e-chuck) to secure the mask.

FIG. 1B is a simplified schematic diagram of a detail of an extreme ultraviolet lithography tool according to an embodiment of the disclosure showing the exposure of photoresist coated substrate 210 secured on a substrate stage 208 of the exposure device 200 with a patterned beam of EUV light. The exposure device 200 is an integrated circuit lithography tool such as a stepper, scanner, step and scan system, direct write system, device using a contact and/or proximity mask, etc., provided with one or more optics 205a, 205b, for example, to illuminate a patterning optic 205c, such as a reticle, with a beam of EUV light, to produce a patterned beam, and one or more reduction projection optics 205d, 205e, for projecting the patterned beam onto the photoresist coated substrate 210. A mechanical assembly (not shown) may be provided for generating a controlled relative movement between the photoresist coated substrate 210 and the patterning optic 205c. As further shown in FIG. 2, the EUVL tool includes an EUV radiation source 100 including an EUV light radiator ZE emitting EUV light in a chamber 105 that is reflected by a collector 110 along a path into the exposure device 200 to irradiate the photoresist coated substrate 210.

As used herein, the term “optic” is meant to be broadly construed to include, and not necessarily be limited to, one or more components which reflect and/or transmit and/or operate on incident light, and includes, but is not limited to, one or more lenses, windows, filters, wedges, prisms, grisms, gradings, transmission fibers, etalons, diffusers, homogenizers, detectors and other instrument components, apertures, axicons and mirrors including multi-layer mirrors, near-normal incidence mirrors, grazing incidence mirrors, specular reflectors, diffuse reflectors and combinations thereof. Moreover, unless otherwise specified, the term “optic”, as used herein, is directed to, but not limited to, components which operate solely or to advantage within one or more specific wavelength range(s) such as at the EUV output light wavelength, the irradiation laser wavelength, a wavelength suitable for metrology or any other specific wavelength.

In various embodiments of the present disclosure, the photoresist coated substrate 210 is a semiconductor wafer, such as a silicon wafer or other type of wafer to be patterned. The EUVL tool further includes other modules or is integrated with (or coupled with) other modules in some embodiments.

As shown in FIG. 1A, the EUV radiation source 100 includes a target droplet generator 115 and a collector 110, enclosed by a chamber 105. For example, the collector 110 is a laser-produced plasma (LPP) collector. In various embodiments, the target droplet generator 115 includes a reservoir to hold a source material and a nozzle 120 through which target droplets DP of the source material are supplied into the chamber 105.

In some embodiments, the target droplets DP are metal droplets of tin (Sn), lithium (Li), or an alloy of Sn and Li. In some embodiments, the target droplets DP each have a diameter in a range from about 10 microns (μm) to about 100 μm. For example, in an embodiment, the target droplets DP are tin droplets, having a diameter of about 10 μm to about 100 μm. In other embodiments, the target droplets DP are tin droplets having a diameter of about 25 μm to about 50 μm. In some embodiments, the target droplets DP are supplied through the nozzle 120 at a rate in a range from about 50 droplets per second (i.e., an ejection-frequency of about 50 Hz) to about 50,000 droplets per second (i.e., an ejection-frequency of about 50 kHz).

Referring back to FIG. 1A, an excitation laser LR2 generated by the excitation laser source 300 is a pulse laser. The laser pulses LR2 are generated by the excitation laser source 300. The excitation laser source 300 may include a laser generator 310, laser guide optics 320 and a focusing apparatus 330. In some embodiments, the laser generator 310 includes a carbon dioxide (CO2) or a neodymium-doped yttrium aluminum garnet (Nd:YAG) laser source with a wavelength in the infrared region of the electromagnetic spectrum. For example, the laser generator 310 has a wavelength of about 9.4 μm or about 10.6 μm, in an embodiment. The laser light LR1 generated by the laser generator 310 is guided by the laser guide optics 320 and focused into the excitation laser LR2 by the focusing apparatus 330, and then introduced into the EUV radiation source 100.

In some embodiments, the excitation laser LR2 includes a pre-heat laser and a main laser. In such embodiments, the pre-heat laser pulse (interchangeably referred to herein as the “pre-pulse”) is used to heat (or pre-heat) a given target droplet to create a low-density target plume with multiple smaller droplets, which is subsequently heated (or reheated) by a pulse from the main laser, generating increased emission of EUV light.

In various embodiments, the pre-heat laser pulses have a spot size about 100 μm or less, and the main laser pulses have a spot size in a range of about 150 μm to about 300 μm. In some embodiments, the pre-heat laser and the main laser pulses have a pulse-duration in the range from about 10 ns to about 50 ns, and a pulse-frequency in the range from about 1 kHz to about 100 kHz. In various embodiments, the pre-heat laser and the main laser have an average power in the range from about 1 kilowatt (kW) to about 50 kW. The pulse-frequency of the excitation laser LR2 is matched with (e.g., synchronized with) the ejection-frequency of the target droplets DP in an embodiment.

The excitation laser LR2 is directed through windows (or lenses) into the zone of excitation ZE in front of the collector 110. The windows are made of a suitable material substantially transparent to the laser beams. The generation of the pulse lasers is synchronized with the ejection of the target droplets DP through the nozzle 120. As the target droplets move through the excitation zone, the pre-pulses heat the target droplets and transform them into low-density target plumes. A delay between the pre-pulse and the main pulse is controlled to allow the target plume to form and to expand to an optimal size and geometry. In various embodiments, the pre-pulse and the main pulse have the same pulse-duration and peak power. When the main pulse heats the target plume, a high-temperature plasma is generated. The plasma emits EUV radiation EUV, which is collected by the collector 110. The collector 110 further reflects and focuses the EUV radiation for the lithography exposing processes performed through the exposure device 200. The droplet catcher 125 is used for catching excessive target droplets. For example, some target droplets may be purposely missed by the laser pulses.

In some embodiments, the collector 110 is designed with a proper coating material and shape to function as a mirror for EUV collection, reflection, and focusing. In some embodiments, the collector 110 is designed to have an ellipsoidal geometry. In some embodiments, the coating material of the collector 110 is similar to the reflective multilayer of the EUV mask. In some examples, the coating material of the collector 110 includes a ML (such as a plurality of Mo/Si film pairs) and may further include a capping layer (such as Ru) coated on the ML to substantially reflect the EUV light. In some embodiments, the collector 110 may further include a grating structure designed to effectively scatter the laser beam directed onto the collector 110. For example, a silicon nitride layer is coated on the collector 110 and is patterned to have a grating pattern.

In the present disclosure, the terms mask, photomask, and reticle are used interchangeably. In the present embodiment, the patterning optic 205c is a reflective mask 205c. The reflective mask 205c also includes a reflective ML deposited on the substrate. The ML includes a plurality of film pairs, such as molybdenum-silicon (Mo/Si) film pairs (e.g., a layer of molybdenum above or below a layer of silicon in each film pair). Alternatively, the ML may include molybdenum-beryllium (Mo/Be) film pairs, or other suitable materials that are configurable to highly reflect the EUV light.

The mask 205c may further include a capping layer, such as ruthenium (Ru), disposed on the ML for protection. The mask 205c further includes an absorption layer deposited over the ML. The absorption layer is patterned to define a layer of an integrated circuit (IC), the absorber layer is discussed below in greater detail according to various aspects of the present disclosure. Alternatively, another reflective layer may be deposited over the ML and is patterned to define a layer of an integrated circuit, thereby forming a EUV phase shift mask.

The mask 205c and the method making the same are further described in accordance with some embodiments. In some embodiments, the mask fabrication process includes two operations: a blank mask fabrication process and a mask patterning process. During the blank mask fabrication process, a blank mask is formed by deposing suitable layers (e.g., reflective multiple layers) on a suitable substrate. The blank mask is then patterned during the mask patterning process to achieve a desired design of a layer of an integrated circuit (IC). The patterned mask is then used to transfer circuit patterns (e.g., the design of a layer of an IC) onto a semiconductor wafer. The patterns can be transferred over and over onto multiple wafers through various lithography processes. A set of masks is used to construct a complete IC.

One example of the reflective mask 205c is shown in FIG. 2. The reflective mask 205c in the illustrated embodiment is a EUV mask, and includes a substrate 30 made of a LTEM. The LTEM material may include TiO₂ doped SiO₂, and/or other low thermal expansion materials known in the art. In some embodiments, a conductive layer 32 is additionally disposed under on the backside of the LTEM substrate 30 for the electrostatic chucking purpose. In one example, the conductive layer 32 includes chromium nitride (CrN), though other suitable compositions are possible.

The reflective mask 205c includes a reflective multilayer (ML) structure 34 disposed over the LTEM substrate 30. The ML structure 34 may be selected such that it provides a high reflectivity to a selected radiation type/wavelength. The ML structure 34 includes a plurality of film pairs, such as Mo/Si film pairs (e.g., a layer of molybdenum above or below a layer of silicon in each film pair). Alternatively, the ML structure 34 may include Mo/Be film pairs, or any materials with refractive index difference being highly reflective at EUV wavelengths.

Still referring to FIG. 2, the EUV mask 205c also includes a capping layer 36 disposed over the ML structure 34 to prevent oxidation of the ML. The EUV mask 205c may further include a buffer layer 38 disposed above the capping layer 36 to serve as an etching-stop layer in a patterning or repairing process of an absorption layer, which will be described later. The buffer layer 38 has different etching characteristics from the absorption layer disposed thereabove. The buffer layer 38 includes ruthenium (Ru), Ru compounds such as RuB, RuSi, chromium (Cr), chromium oxide, and chromium nitride in various examples.

The EUV mask 205c also includes an absorber layer 40 (also referred to as an absorption layer) formed over the buffer layer 38. In some embodiments, the absorber layer 40 absorbs the EUV radiation directed onto the mask. In various embodiments, the absorber layer may be made of tantalum boron nitride (TaBN), tantalum boron oxide (TaBO), or chromium (Cr), Radium (Ra), or a suitable oxide or nitride (or alloy) of one or more of the following materials: Actium, Radium, Tellurium, Zinc, Copper, and Aluminum.

FIGS. 3, 6, 11, 12A, 12B, 13A and 13B are diagrammatic fragmentary cross-sectional side views of a semiconductor device 45 at various stages of fabrication in accordance with various aspects of the present disclosure. In some embodiments, the semiconductor device 45 may include an integrated circuit (IC) chip, system on chip (SoC), or portion thereof, and may include various passive and active microelectronic devices such as resistors, capacitors, inductors, diodes, metal-oxide semiconductor field effect transistors (MOSFET), complementary metal-oxide semiconductor (CMOS) transistors, bipolar junction transistors (BJT), laterally diffused MOS (LDMOS) transistors, high power MOS transistors, or other types of transistor.

Reference is made to FIG. 3. A photoresist layer 15 is coated on a surface of a layer to be patterned (or target layer) or a substrate in an operation S100. For example, the semiconductor device 45 including a substrate 13 is illustrated. In some embodiments, the substrate 13 is a silicon substrate doped with a p-type dopant such as boron (for example a p-type substrate). Alternatively, the substrate 13 could be another suitable semiconductor material. For example, the substrate 13 may be a silicon substrate that is doped with an n-type dopant such as phosphorous or arsenic (an n-type substrate). The substrate 13 could include other elementary semiconductors such as germanium and diamond. The substrate 13 could optionally include a compound semiconductor and/or an alloy semiconductor. Further, the substrate 13 could include an epitaxial layer (epi layer), may be strained for performance enhancement, and may include a silicon-on-insulator (SOI) structure.

In some embodiments, the substrate 13 is substantially conductive or semi-conductive. The electrical resistance may be less than about 103 ohm-meter. In some embodiments, the substrate 13 contains metal, metal alloy, or metal nitride/sulfide/selenide/oxide/silicide with the formula MXₐ, where M is a metal, and X is N, S, Se, O, Si, and where “a” is in a range from about 0.4 to about 2.5. For example, the substrate 13 may contain Ti, Al, Co, Ru, TiN, WN₂, or TaN.

In some other embodiments, the substrate 13 contains a dielectric material with a dielectric constant in a range from about 1 to about 40. In some other embodiments, the substrate 13 contains Si, metal oxide, or metal nitride, where the formula is MXb, wherein M is a metal or Si, and X is N or O, and wherein “b” is in a range from about 0.4 to 2.5. For example, the substrate 13 may contain SiO₂, silicon nitride, aluminum oxide, hafnium oxide, or lanthanum oxide.

The photoresist layer 15 may be formed by a spin-coating process. When exposed to actinic radiation, the photoresist layer 15 undergoes one or more chemical reactions causing a change in solubility in a developer composition. Formation of the photoresist layer 15 may include depositing a photoresist composition on the substrate 13 to form the photoresist layer 15. In certain embodiments, the photoresist composition includes a polymer, a photo acid generator (PAG), a quencher and a solvent. The quencher may include a base that scavenges trace acids, while not having an excessive impact on the performance of the photoresist composition. In some other embodiments, the photoresist composition may include surfactant, chromophore, cross linker, the like, or a combination thereof. In some embodiments, the polymer has a molecular weight in a range from about 1000 to about 10000.

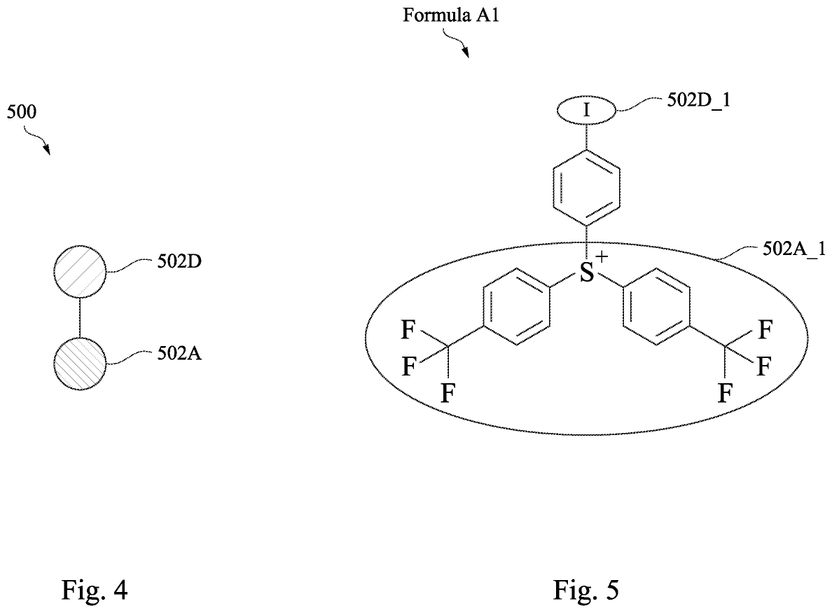

FIG. 4 is a schematic diagram of a photoactive cation 500 of the PAG in accordance with some embodiments. In some embodiments, the PAG includes the photoactive cation 500 and an anion. The photoactive cation 500 includes one or more high EUV absorbing groups 502D for EUV absorbing and a donor-acceptor (D-A) type structure for EUV-induced intramolecular charge transfer. For example, the PAG has a moiety 502A attached to the high EUV absorbing groups 502D in which the high EUV absorbing groups 502D acts as a donor and the moiety 502A acts as an acceptor. In other words, the high EUV absorbing group 502D and a donor-acceptor design are included in the same molecule of the PAG. The high EUV absorbing group 502D can include one or more atoms which have a high EUV absorbing cross-section. In some embodiments, the high EUV absorbing group 502D includes I, Sn, Xe, Te, Sb, In, Cs, At, Bi, Po or atoms with an EUV cross section being higher than about 1×10⁷ cm²/mol. If the EUV cross section of the high EUV absorbing group 502D is lower than about 1×10⁷ cm²/mol, a desired EUV sensitivity of the PAG may not be achieved. FIG. 5 is an example structural formula (A1) of the photoactive cation 500 of the PAG in accordance with some embodiments. An I atom in the formula (A1) acts as a high EUV absorbing group 502D_1, and an onium salt in the formula (A1) acts as the moiety 502A_1. The iodine (I) acts as the electron donor because it has strong electron-donating ability afterexcitation. In other words, the iodine (I) is an electron donating group, and the moiety 502A_1 is an electron withdrawing group.

The moiety 502A attached to the high EUV absorbing group 502D is for photo acid generation in which the moiety 502A is selected from a group consisting of onium salts, selenium salts, phosphonium salts, iodonium salts and sulfonium salts. The moiety 502A can be a non-cyclic structure or a cyclic structure in which the cyclic structure can be an aromatic or a non-aromatic ring. The cyclic structure of the moiety 502A can be an aromatic or a non-aromatic ring having a substituted carbon group or functionalized group including —H, —F, —CF₃, —I, —Br, —Cl, —NH₂, —COOH, —OH, —SH, —N₃, —S(═O)—, alkene, alkyne, imine, ether, ester, aldehyde, ketone, amide, sulfone, acetic acid, cyanide, allene, alcohol, amine, phosphine, phosphite, aniline, pyridine, or pyrrole. Examples of the PAG include formulae (A1) to (A12):

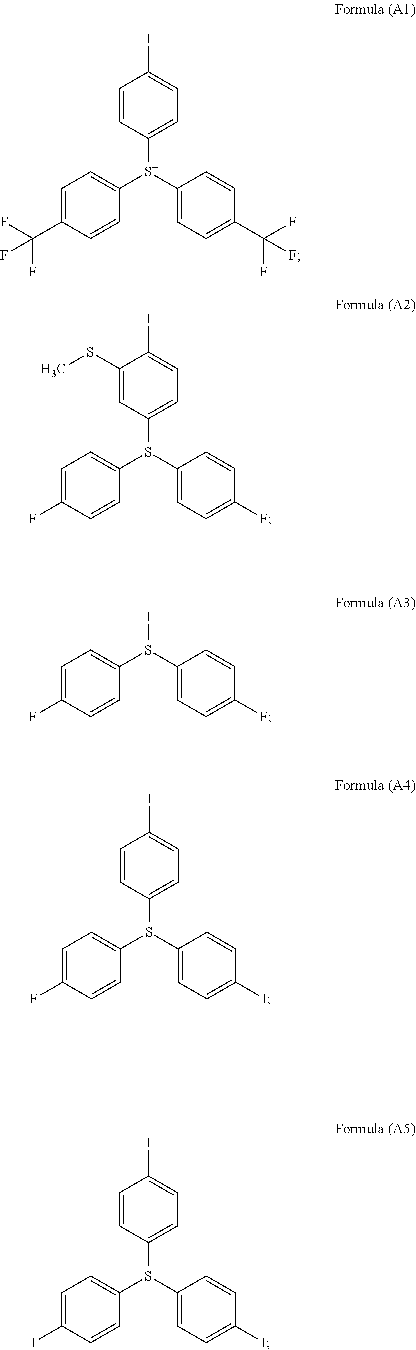 [SMILES: FC(F)(F)c1ccc([S+](c2ccc(I)cc2)c2ccc(C(F)(F)F)cc2)cc1 ; CSc1cc([S+](c2ccc(F)cc2)c2ccc(F)cc2)ccc1I ; Fc1ccc([S+](I)c2ccc(F)cc2)cc1 ; Fc1ccc([S+](c2ccc(I)cc2)c2ccc(I)cc2)cc1 ; Ic1ccc([S+](c2ccc(I)cc2)c2ccc(I)cc2)cc1]

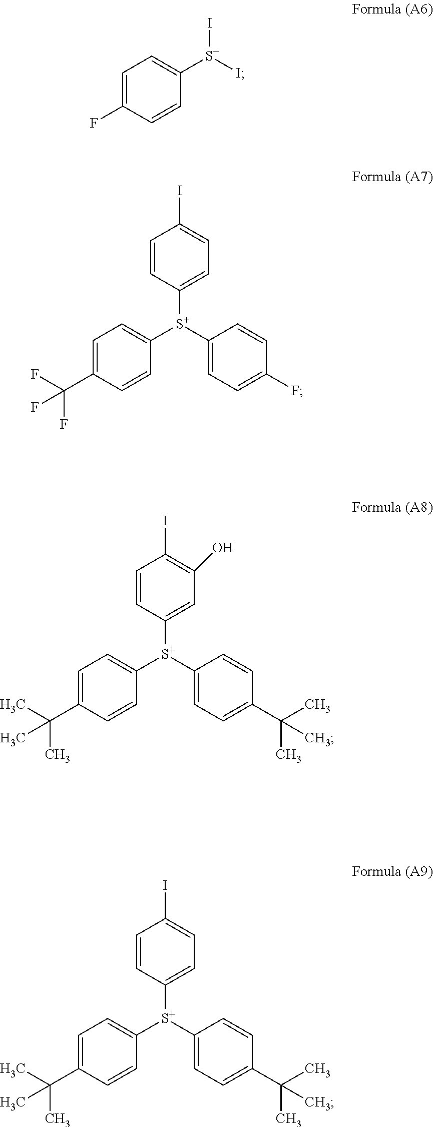 [SMILES: Fc1ccc([S+](I)I)cc1 ; Fc1ccc([S+](c2ccc(I)cc2)c2ccc(C(F)(F)F)cc2)cc1 ; CC(C)(C)c1ccc([S+](c2ccc(C(C)(C)C)cc2)c2ccc(I)c(O)c2)cc1 ; CC(C)(C)c1ccc([S+](c2ccc(I)cc2)c2ccc(C(C)(C)C)cc2)cc1]

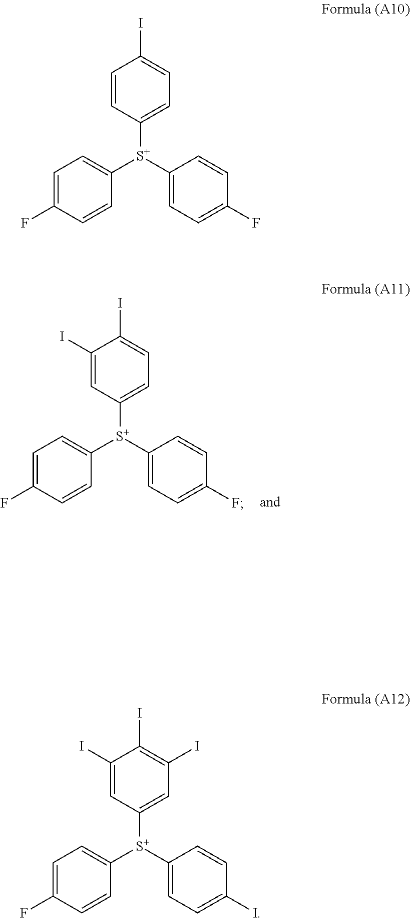 [SMILES: Fc1ccc([S+](c2ccc(F)cc2)c2ccc(I)cc2)cc1 ; Fc1ccc([S+](c2ccc(F)cc2)c2ccc(I)c(I)c2)cc1 ; Fc1ccc([S+](c2ccc(I)cc2)c2cc(I)c(I)c(I)c2)cc1]

The high EUV absorbing group 502D can absorb an EUV photon due to its inner-shell electrons and have a rapid intramolecular charge transfer from itself to the moiety 502A, which will be discussed in greater detail below.

In some embodiments, the photoresist composition can be organic or inorganic. For example, the photoresist composition may be inorganic and include at least a metal complex, metal cluster, PAG, quencher (or base), and solvent. The photoresist composition may include a metallic photoresist being a negative tone photoresist. In some embodiments, the metallic photoresist may include a surfactant, chromophore, ligands, and counter ions. For example, the polymer of the negative tone metallic photoresist includes at least one of metal complexes with several ligands and counter ions. The ligands can dissociate after an exposure and further complexation or condensation may happen via oxygen treatment using such as H₂O, O₂, or a combination thereof. After the polymer cross links or undergoes complexation or condensation, the polymer become more hydrophobic.

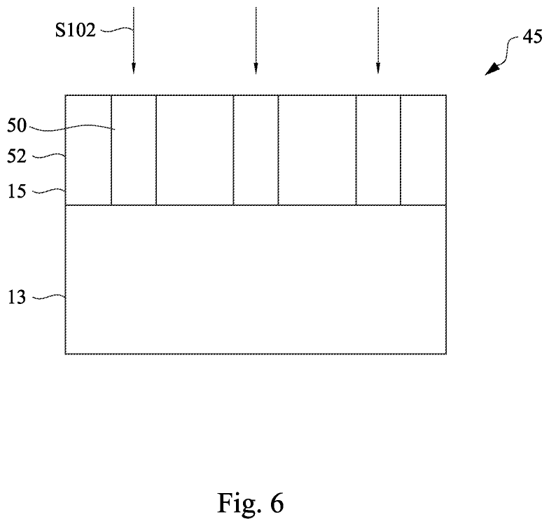

Referring to FIG. 6, after forming the photoresist layer 15, the photoresist layer 15 is exposed to an actinic radiation S102. For example, the photoresist layer 15 has an exposed region 50 and an unexposed region 52. In some embodiments, the photoresist layer 15 is exposed to the actinic radiation S102 having a wavelength being substantially less than about 250 nm which comprises KrF, ArF, EUV, E-beam, or a combination thereof. Due to the actinic radiation S102, the PAG generate strong acids which can be sulfonic acid, fluorosulfonic acid or other strong acids which pKa <0. The PAG may generate an acid through a plurality of reactions, for example, electron generation, acid “precursor” formation, anion release and acid release.

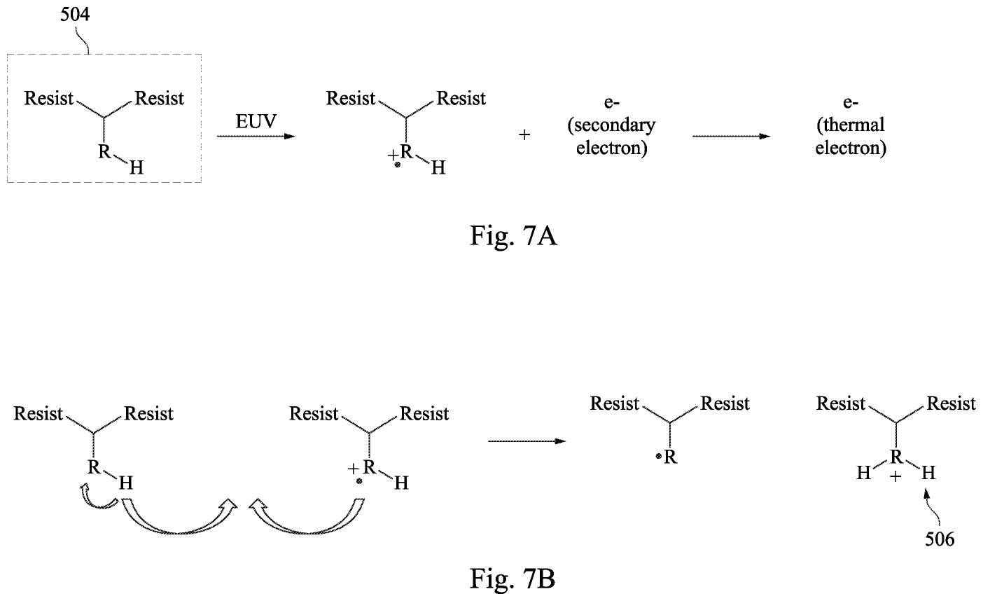

FIG. 7A shows an electron generation of a polymer 504 of the photoresist composition upon an EUV radiation in accordance with some embodiments. FIG. 7B shows formation of an acid “precursor” 506 of the polymer of the photoresist composition in accordance with some embodiments. The PAG can decompose via at least two charge transfer mechanisms for anion release. The at least two charge transfer mechanisms include an intermolecular charge transfer mechanism and an intramolecular charge transfer mechanism. Since the PAG includes both of the intermolecular charge transfer and the intramolecular charge transfer, an acid generation efficiency is improved, which is beneficial for improving lithography performance.

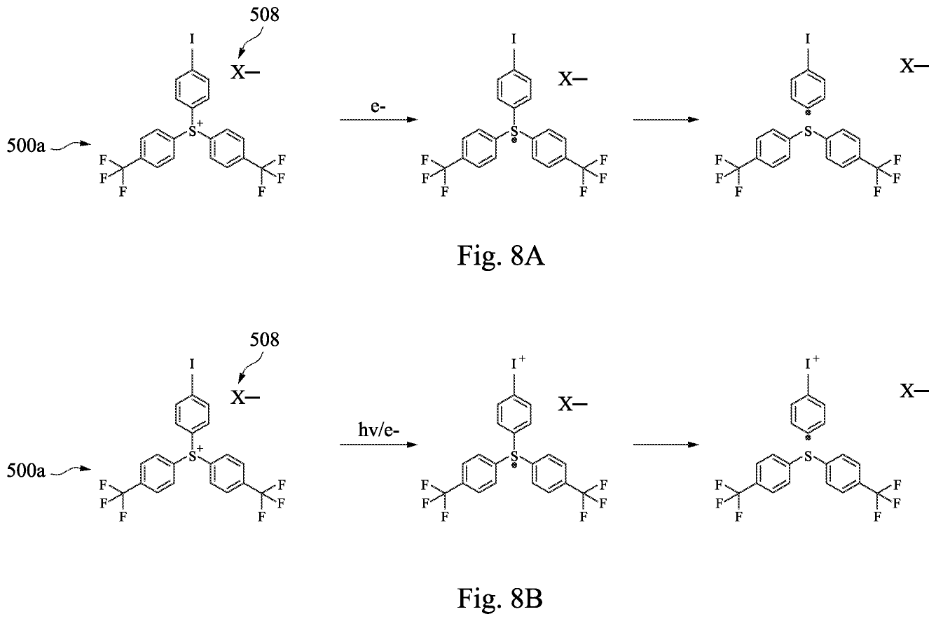

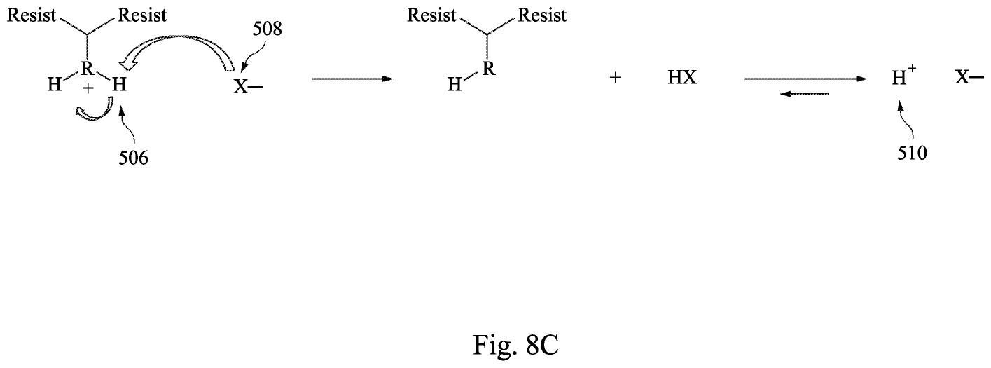

FIG. 8A shows an intermolecular charge transfer mechanism of the PAG in accordance with some embodiments. FIG. 8B shows an intramolecular charge transfer mechanism of the PAG in accordance with some embodiments. In FIG. 8A, the PAG includes a photoactive cation 500a attached to an anion 508. Reference is made to FIGS. 7A, 7B and 8A. In the intermolecular charge transfer mechanism, when the photoresist composition is irradiated by EUV, the polymer 504 can generate secondary electrons and then form acid “precursors” 506. Since the initially formed secondary electrons with high energy, for example, in a range from about 78 eV to about 86 eV, cannot be directly used by the photoactive cation 500 (see FIG. 4) of the PAG, some energy loss mechanisms is required for such secondary electrons to reduce their energy to be below about 20 eV, which is referred to as a thermal electron (see FIG. 7A). When the secondary electrons have energy low enough, they can react and combine with the photoactive cation 500a to make the photoactive cation 500a decompose and release the anion 508. FIG. 8C shows an acid release in accordance with some embodiments. Referring to FIG. 8C, the released anion 508 can trigger the acid “precursor” 506 to release an acid 510 and the acid 510 can be further used for chemically amplified reactions.

Referring back to FIG. 8B, in the intramolecular charge transfer mechanism, when the PAG is irradiated by EUV (in which an absorbing energy is represented by E=hν (E: energy, h: Plank constant, ν: wavelength), since the photoactive cation 500a includes one or more high EUV absorbing groups for EUV absorbing, which is an I atom in this example, and the donor-acceptor (D-A) type structure for EUV-induced intramolecular charge transfer, the EUV-induced electron transition is more efficiently achieved. The PAG can decompose via the at least two charge transfer mechanisms for anion release in which the at least two charge transfer mechanisms include the intermolecular charge transfer mechanism and the intramolecular charge transfer mechanism. The intramolecular charge transfer mechanism is faster than the intermolecular charge transfer mechanism, which is advantageous for improving the efficiency of the anion release. Therefore, acid generation of the PAG by EUV is increased. Secondary electron loss is reduced by intramolecular charge transfer mechanism.

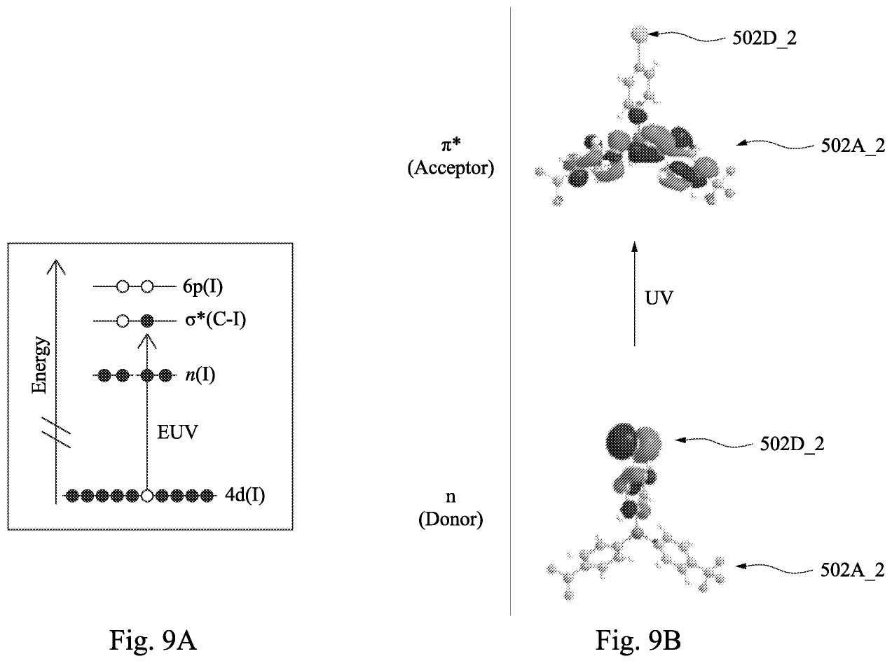

FIG. 9A shows a graph of EUV absorbing of CH₃—I group of the PAG in accordance with some embodiments. Reference is made to FIGS. 8B and 9A. For example, the high EUV absorbing group is iodine (I) in which iodine has 4d orbital electrons and can absorb the EUV light. Iodine can generate a large amount of secondary electrons including 4d and Auger electrons. An intramolecular electronic transition may occur in the CH₃—I (or C—I) group by the EUV light after the iodine absorbs the EUV light. A molecular electronic transition takes place when electrons in a molecule are excited from one energy level to higher energy level. For example, electrons occupying an EUV sensitive molecular orbital is 4d orbital for the iodine (I), and can get excited to an antibonding molecular orbital of a sigma bond (σ). In some embodiments, the intramolecular charge transfer includes d-σ* transition and d-D*-σ* transition, in which the d stands for 4d orbital, the σ* stands for sigma bond excitation state, and the D* stands for donor excitation state.

FIG. 9B is a schematic diagram of highest occupied molecular orbital (HOMO) and lowest unoccupied molecular orbital (LUMO) under UV absorption of the PAG in accordance with some embodiments. In some embodiments, when the actinic radiation S102 (see FIG. 6) is UV light, the PAG can have an n-π* transition at the UV light. The high EUV absorbing groups 502D_2 is iodine (I) and can be an electron donating group, and the moiety 502A_2 is an electron withdrawing group. Because the PAG has the high EUV absorbing groups, a large amount of secondary electrons can be generated. Due to the donor-acceptor (D-A) type structure, efficient EUV-induced electronic transition can be achieved.

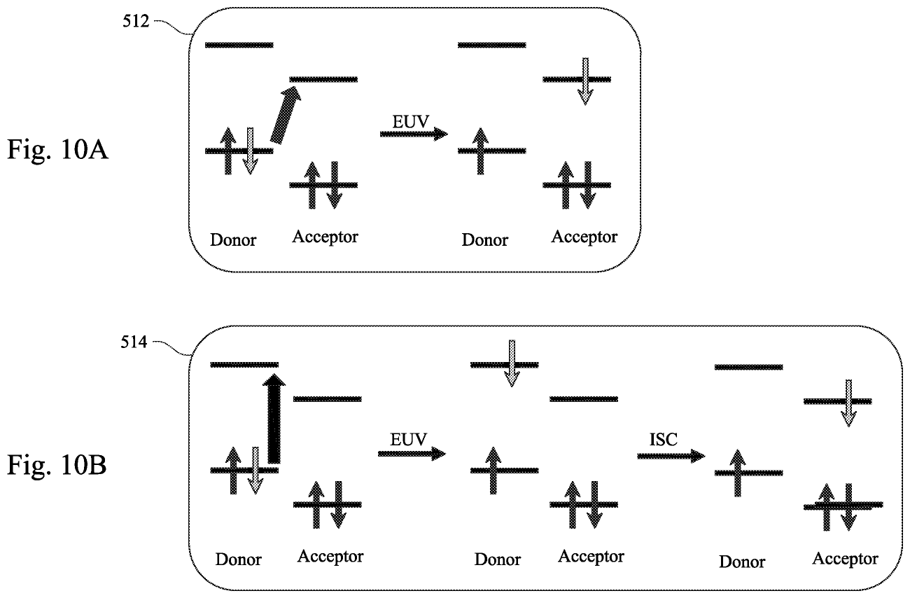

FIGS. 10A and 10B are diagrams of a first mechanism 512 and a second mechanism 514 of the intramolecular charge transfer between donor and acceptor by EUV, respectively, in accordance with some embodiments. In FIG. 10A, in the first mechanism 512, an electron is directly excited from a ground state of the donor to an excited state of the acceptor by the EUV light. In FIG. 10B, in the second mechanism 514, an electron is directly excited from a ground state to an excited state of the donor and then follows by intersystem crossing (ISC) to transfer the electron from the excited state of the donor to the excited state of the acceptor.

In some embodiments, after the photoresist layer 15 is exposed to the actinic radiation S102, a post exposure (PEB) process is performed to the photoresist layer 15. In some embodiments, the photoresist composition is a positive tone photoresist, and the polymer thereof is an acid cleavable polymer. The PAG release acid after the actinic radiation S102, and the acid cleaves the polymer in the post exposure baking process. After the acid cleaves the polymer, the polymer becomes more hydrophilic. In greater detail, after the PEB process, the strong acids (e.g., the acid in FIG. 8C) may deprotect acid-labile groups (ALGs) in the exposed region 50 of the photoresist layer 15, and the polarity of the polymer transfers from hydrophobic to hydrophilic.

In some embodiments where the photoresist composition includes the metallic polymer, after the actinic radiation S102, the metallic polymer dissociates some ligands and gives some active empty sites. After the PEB, the empty sites are complexed with H₂O or O₂ in the exposed region 50, and the structure of the metallic polymer becomes huge metal oxides.

In some embodiments, the photoresist layer 15 is a negative tone photoresist. For example, the negative tone photoresist includes at least one of an acid catalyzed cross linkable polymer or polymeric pynacol. The PAG releases acid after the actinic radiation S102, and the acid catalyze the cross linking reaction of the acid catalyzed cross linkable polymer or make the polymeric pinacol undergo pinacol rearrangement. After the polymer cross link or undergo pinacol rearrangement, the polymer becomes more hydrophobic.

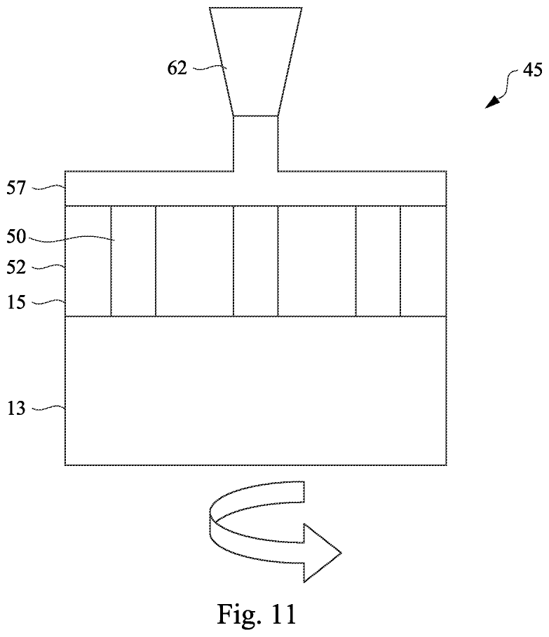

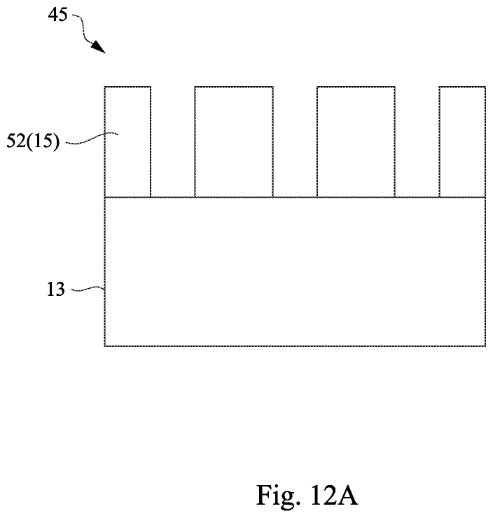

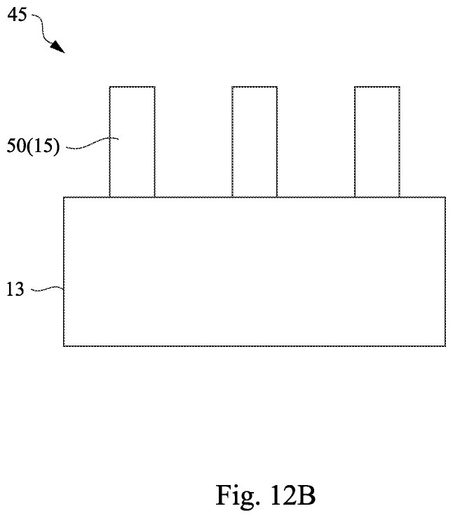

The photoresist layer 15 is subsequently developed by applying a developer to the photoresist layer 15, as shown in FIG. 11, a developer 57 is supplied from a dispenser 62 to the photoresist layer 15. In some embodiments, the exposed region 50 of the photoresist layer 15 is removed by the developer 57, as shown in FIG. 12A. For example, in some embodiments where the photoresist composition is the positive tone photoresist, after the polymer becomes more hydrophilic, the polymer can be dissolved by the developer 57 made of a basic solution, for example, tetramethylammonium hydroxide (TMAH) aqueous solution, such as a 2.38% TMAH. In some embodiments, the developer 57 is made of an organic solvent, for example, normal butyl acetate (also known as n-butyl acetate). After the polymer becomes more hydrophilic, the polymer cannot be dissolved by this organic solvent such that the exposed region 50 remains on the substrate 13, and the unexposed region 52 is removed, as shown in FIG. 12B. Still referring to FIG. 12B, in some embodiments where the photoresist composition is the negative tone photoresist, the developer 57 (see FIG. 11) may be a basic solution. After the polymer becomes more hydrophobic, the polymer cannot be dissolved by the basic solution, for example, tetramethylammonium hydroxide (TMAH) aqueous solution, such as a 2.38% TMAH. In some other embodiments, the developer 57 (see FIG. 11) may be made of an organic solvent, for example, normal butyl acetate (also known as n-butyl acetate). After the polymer becomes a polymer with an increased molecular weight (MW), the polymer cannot be dissolved in the organic solvent.

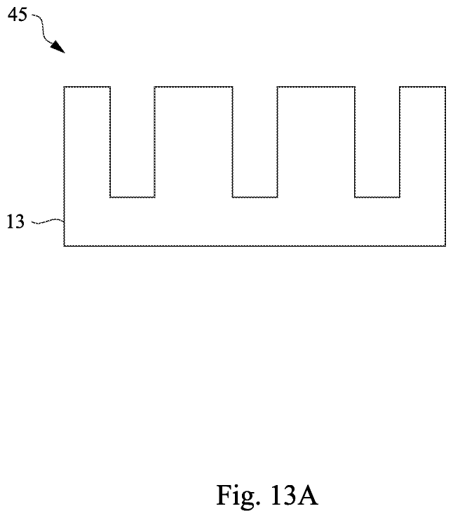

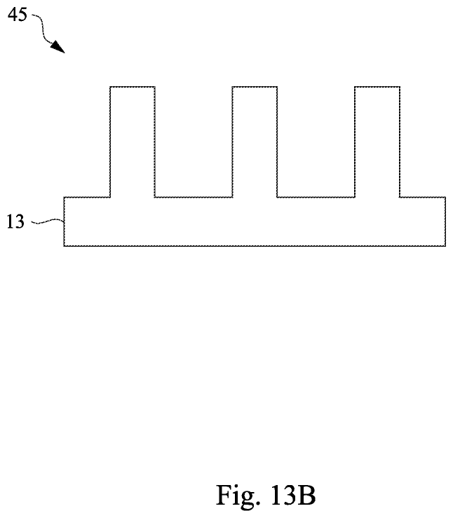

In some embodiments, a pattern of the developed photoresist layer 15 is extended into the layer to be patterned or the substrate 13 to create a pattern in the substrate 13, thereby transferring the pattern in the photoresist layer 15 into the substrate 13, as shown in FIGS. 13A and 13B. The pattern is extended into the substrate 13 by an etching operation, using one or more suitable etchants. A remaining portion of the photoresist layer 15 is at least partially removed during the etching operation in some embodiments. In other embodiments, the remaining photoresist layer 15 is removed after etching the substrate 13 by using a suitable photoresist stripper solvent or by a photoresist ashing operation.

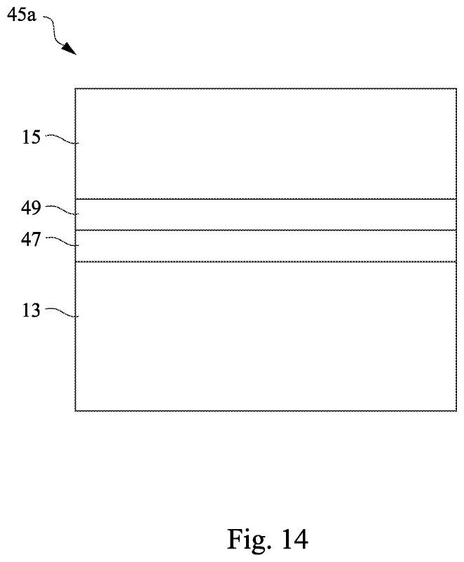

FIGS. 14-19 are diagrammatic fragmentary cross-sectional side views of a semiconductor device 45a at various stages of fabrication in accordance with various aspects of the present disclosure. Reference is made to FIG. 14. The semiconductor device 45a is similar to the semiconductor device 45 as discussed previously with regard to FIG. 3, except for the semiconductor device 45a further including a first material layer 47 an a second material layer 49. The first material layer 47, the second material layer 49 and the photoresist layer 15 are formed on the substrate 13 in sequence. In some embodiments, the semiconductor device 45a may include other extra layers (not shown) on the substrate 13. In some embodiments, the first material layer 47 has a different optical property than a silicon hard mask, an extra layer, and the photoresist layer 15. For example, the first material layer 47 has a substantially different n, k, T value (thickness) than that of the photoresist layer 15. In some embodiments, the first material layer 47 can be a CVD or PVD deposited film. In some embodiments, the first material layer 47 includes at least one of different polymer structure, acid-labile molecule, PAG loading, quencher loading, chromophore, cross linker, or solvent, which lead to the different n value than the photoresist layer 15. The first material layer 47 and the photoresist layer 15 have etch resistance. The first material layer 47 and the photoresist layer 15 may contain at least one of etch resistance molecule, for example, molecule including low onishi number structure, double bond, triple bond, silicon, silicon nitride, Ti, TiN, Al, aluminum oxide, SiON, or the like.

In some embodiments, the second material layer 49 includes at least a silicon-containing polymer, metalline polymer, organic polymer, metal complexes, metal oxides, metal nanoparticles, crosslinker, chromophore, PAG, quencher (or base), fluoro additive, and solvent. In some embodiments, the second material layer 49 may be referred to as a silicon hard mask layer. The second material layer 49 may have a different optical property than the first material layer 47 and the photoresist layer 15. The second material layer 49 has substantially different n, k or T value than the n, k or T values of the first material layer 47 and the photoresist layer 15. For example, as compared to the photoresist layer 15 and the first material layer 47, the second material layer 49 includes at least one of different silicon-containing polymer structure, acid-labile molecule, PAG loading, quencher loading, chromophore, cross linker, or solvent, which lead to the different n value than the photoresist layer 15. In some embodiments, the first material layer 47, the second material layer 49 and the photoresist layer 15 have different etch resistances. The second material layer 49 contains at least one of etching resistance molecule, for example, the molecule including low onishi number structure, double bond, triple bond, silicon, silicon nitride, Ti, TiN, Al, aluminum oxide, SiON, or the like.

In some embodiments where the second material layer 49 is a silicon-containing polymer, the silicon-containing polymer has at least one kind of silicon-containing monomer, such as the silicon-containing monomer contains 0 to about 4 oxygen atoms on the silicon atom, the silicon-containing monomer has 0 to about 4 alkyl groups on the silicon atom in which the alkyl groups attached on the silicon atom can be aliphatic or aromatic groups.

In some embodiments, the second material layer 49 is a blend of silicon-containing polymer and metalline polymer in which the metalline polymer has at least one kind of metal atoms which can absorb EUV light. For example, the metal atom of the metalline polymer can be Hf, Zr, Ti, Cr, W, Mo, Fe, Ru, Os, Co, Rh, Ir, Ni, Pd, Pt, Cu, Ag, Au, Zn, Cd, Al, Ga, Tl, Ge, Sn, Pb, Sb, or Bi. In some embodiments, the second material layer 49 is a silicon polymer blending with metal complexes, metal oxides, and metal nanoparticles. In some embodiments, the metal atom of the metal complexes, metal oxides, and metal nanoparticles can be Hf, Zr, Ti, Cr, W, Mo, Fe, Ru, Os, Co, Rh, Ir, Ni, Pd, Pt, Cu, Ag, Au, Zn, Cd, Al, Ga, Tl, Ge, Sn, Pb, Sb, or Bi.

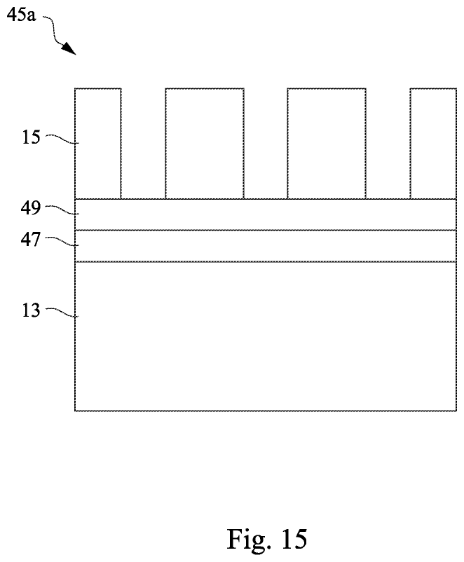

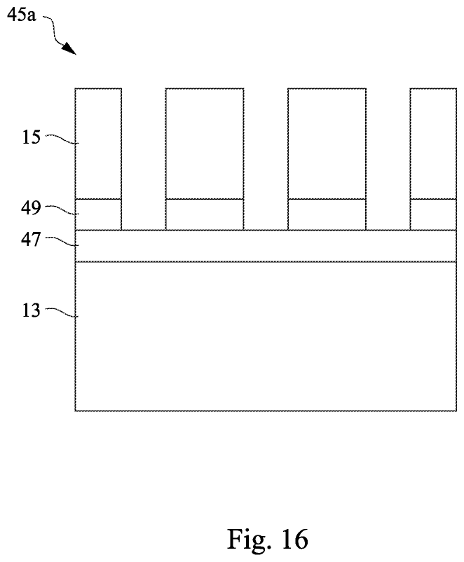

Reference is made to FIG. 15. The photoresist layer 15 is patterned, for example, by exposure and developing processes, as discussed previously with regard to FIGS. 6 and 11-12B, and thus the description thereof is omitted herein. Reference is made to FIG. 16. A first etching process is performed to the second material layer 49 using the patterned photoresist layer 15 as an etch mask, thereby transforming a pattern from the photoresist layer 15 to the second material layer 49, thus forming the patterned second material layer 49. The first etching process is performed using an etchant to selectively etch the second material layer 49 but stops on the first material layer 47. The first etching process may be a dry etch, a wet etch or a combination thereof. After the first etching process, the photoresist layer 15 is removed by wet stripping or plasma ashing.

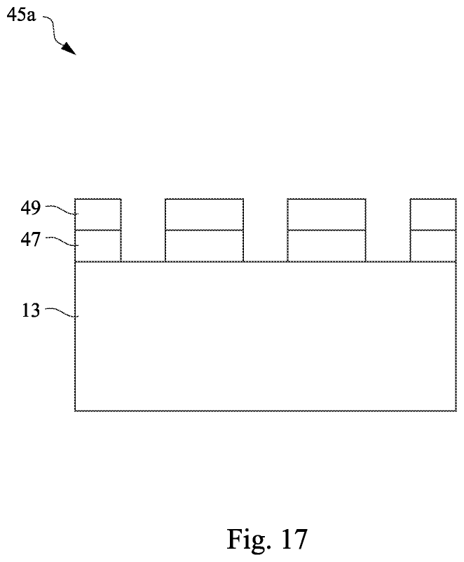

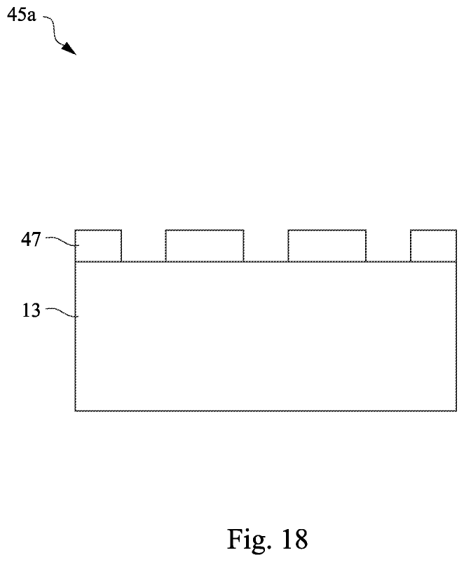

Reference is made to FIG. 17. A second etching process is performed to the first material layer 47 using the patterned second material layer 49 as an etch mask, thereby transforming the pattern from the patterned second material layer 49 to the first material layer 47, forming the patterned first material layer 47. The second etching process is performed using an etchant to selectively etch the first material layer 47 without significantly removal of the patterned second material layer 49 due to the etching selectivity between the second material layer 49 and the first material layer 47. The second etching process may be a dry etch, a wet etch or a combination thereof. After the second etching process, the patterned second material layer 49 may be removed by a proper technique, such as wet etching. The resulting structure is shown in FIG. 18.

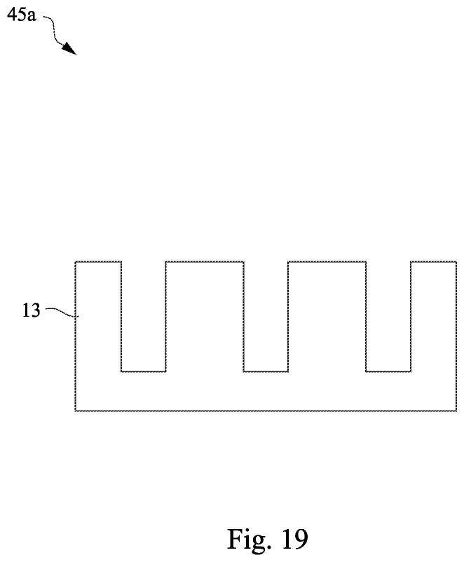

Reference is made to FIG. 19. Using the patterned first material layer (see FIG. 18) as a mask, additional fabrication processes such as etching or implantation may be performed to the substrate 13. For example, the substrate 13 is etched. Thereafter, the patterned first material layer 47 may be removed by a suitable removing process.

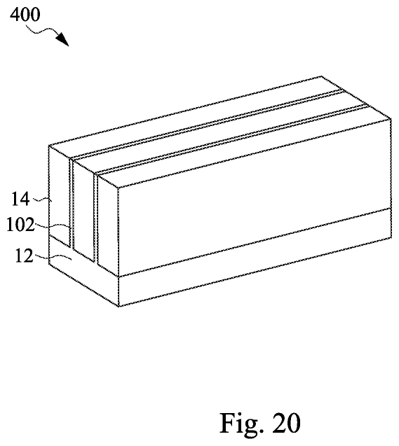

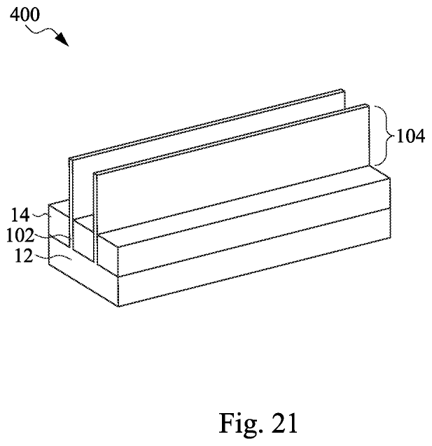

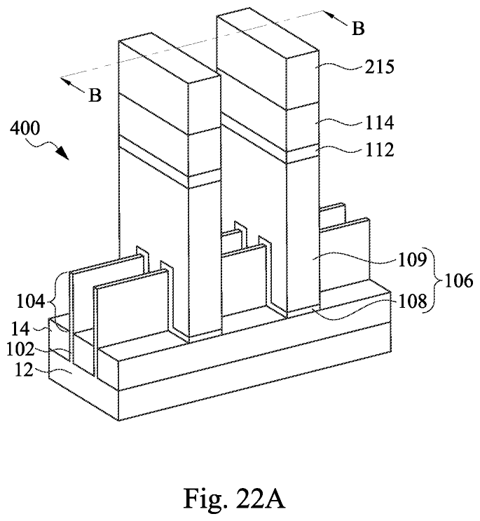

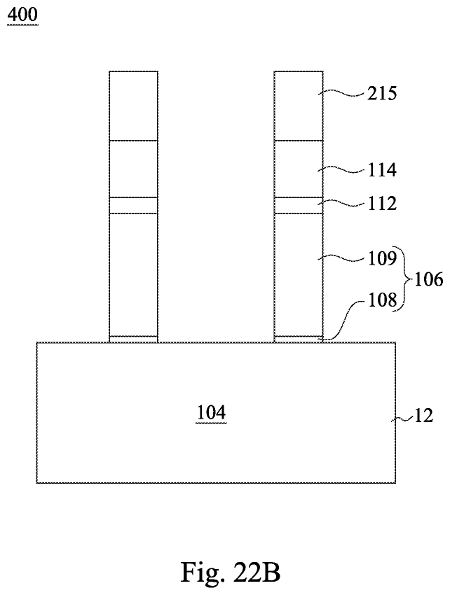

FIGS. 20, 21, and 22A illustrate perspective views of additional fabrication processes in the formation of a semiconductor device 400 on a substrate 12 in accordance with some embodiments of the present disclosure. FIGS. 22B, 23, 24 and 25 illustrate cross-sectional views of additional fabrication processes in the formation of a semiconductor device 400 using a substrate 12 in accordance with some embodiments of the present disclosure. Reference is made to FIG. 21. FIG. 21 illustrates a perspective view of an initial structure. The initial structure includes the substrate 12. The substrate 12 is similar to the substrate 13 in terms of composition and formation, such as being patterned by the photoresist layer 15 as discussed previously with respect to FIGS. 3-13B. Isolation regions such as shallow trench isolation (STI) regions 14 may be formed to extend into the substrate 12. The portions of substrate 12 between neighboring STI regions 14 are referred to as semiconductor strips 102.

The STI regions 14 may include a liner oxide (not shown). The liner oxide may be formed of a thermal oxide formed through a thermal oxidation of a surface layer of the substrate 12. The liner oxide may also be a deposited silicon oxide layer formed using, for example, Atomic Layer Deposition (ALD), High-Density Plasma Chemical Vapor Deposition (HDPCVD), or Chemical Vapor Deposition (CVD). The STI regions 14 may also include a dielectric material over the liner oxide, and the dielectric material may be formed using flowable chemical vapor deposition (FCVD), spin-on coating, or the like.

Referring to FIG. 21, the STI regions 14 are recessed, so that the top portions of semiconductor strips 102 protrude higher than the top surfaces of the neighboring STI regions 14 to form protruding fins 104. The etching may be performed using a dry etching process or a wet etching process.

The materials of fins 104 may also be replaced with materials different from that of substrate 12. For example, if the fins 104 serve for n-type transistors, protruding fins 104 may be formed of Si, SiP, SiC, SiPC, or a III-V compound semiconductor such as InP, GaAs, AlAs, InAs, InAlAs, InGaAs, or the like. On the other hand, if the fins 104 serve for p-type transistors, the protruding fins 104 may be formed of Si, SiGe, SiGeB, Ge, or a III-V compound semiconductor such as InSb, GaSb, InGaSb, or the like.

Referring to FIGS. 22A and 22B, dummy gate structures 106 are formed on the top surfaces and the sidewalls of fins 104. FIG. 22B illustrates a cross-sectional view obtained from a vertical plane containing line B-B in FIG. 22A. Formation of the dummy gate structures 106 includes depositing in sequence a blankly formed gate dielectric layer and a blankly formed dummy gate electrode layer across the fins 104, followed by patterning the blanket formed gate dielectric layer and the blankly formed dummy gate electrode layer. As a result of the patterning, the dummy gate structure 106 includes a dummy gate dielectric layer 108 and a dummy gate electrode 109 over the dummy gate dielectric layer 108. The dummy gate dielectric layers 108 can be any acceptable dielectric layer, such as silicon oxide, silicon nitride, the like, or a combination thereof, and may be formed using any acceptable process, such as thermal oxidation, a spin process, CVD, or the like. The dummy gate electrodes 109 can be any acceptable electrode layer, such as comprising polysilicon, metal, the like, or a combination thereof. The gate electrode layer can be deposited by any acceptable deposition process, such as CVD, plasma enhanced CVD (PECVD), or the like. Each of dummy gate structures 106 crosses over a single one or a plurality of fins 104. The dummy gate structures 106 may have lengthwise directions perpendicular to the lengthwise directions of the respective fins 104.

The blankly formed dummy gate electrode layer and the blankly formed gate dielectric layer may be patterned using a tri-layer structure. Bottom masks 112, top masks 114 and photoresist layers 215, in which the photoresist layers 215 is similar to the photoresist layer 15 with regard to FIG. 3 in terms of composition and formation method, are formed over the blankly formed dummy gate electrode layer in sequence. The above discussion of photoresist layer 15 applies to the photoresist layers 215, unless mentioned otherwise. By using the photoresist layer 215 as a mask, the pattern dimension accuracy of the underlying layer (e.g., the dummy gate electrodes 109 and the dummy gate dielectric layers 108) can be improved.

In an alternative embodiment, the bottom masks 112 and the top masks 114 are made of one or more layers of SiO₂, SiCN, SiON, Al₂O₃, SiN, or other suitable materials. In certain embodiments, the bottom masks 112 include silicon nitride, and the top masks 114 include silicon oxide.

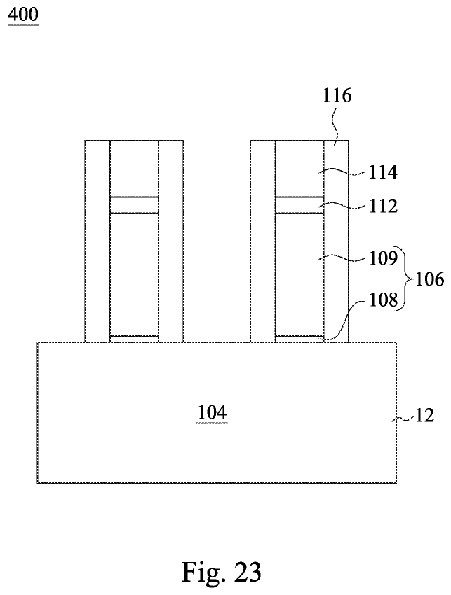

Next, as illustrated in FIG. 23, gate spacers 116 are formed on sidewalls of the dummy gate structures 106. In some embodiments of the gate spacer formation step, a spacer material layer is deposited on the substrate 12. The spacer material layer may be a conformal layer that is subsequently etched back to form gate spacers 116. The spacer material layer is made of a low-k dielectric material. The low-k dielectric material has a dielectric constant (k value) of lower than about 3.5. Suitable materials for the low-k dielectric material may include silicon oxide, silicon nitride, silicon oxynitride, or the like. By way of example and not limitation, the spacer material layer may be formed using processes such as, CVD process, a subatmospheric CVD (SACVD) process, a flowable CVD process, an ALD process, a physical vapor deposition (PVD) process, or other suitable process. An anisotropic etching process is then performed on the deposited spacer material layer to expose portions of the fins 104 not covered by the dummy gate structures 106 (e.g., in source/drain regions of the fins 104). Portions of the spacer material layer directly above the dummy gate structures 106 may be completely removed by this anisotropic etching process. Portions of the spacer material layer on sidewalls of the dummy gate structures 106 may remain, forming gate spacers, which are denoted as the gate spacers 116, for the sake of simplicity. In some embodiments, the gate spacers 116 may be used to offset subsequently formed doped regions, such as source/drain regions. The gate spacers 116 may further be used for designing or modifying the source/drain region profile.

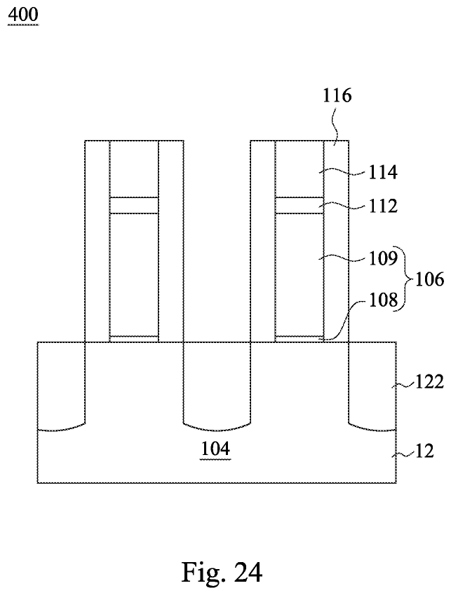

In FIG. 24, after formation of the gate spacers 116 is completed, source/drain epitaxial structures 122 are formed on source/drain regions of the protruding fins 104 that are not covered by the dummy gate structures 106 and the gate spacers 116. In some embodiments, formation of the source/drain epitaxial structures 122 includes recessing source/drain regions of the fin 104, followed by epitaxially growing semiconductor materials in the recessed source/drain regions of the fin 104. The source/drain epitaxial structures 122 are on opposite sides of the dummy gate structure 106.

The source/drain regions of the fins 104 can be recessed using suitable selective etching processing that attacks the fins 104, but hardly attacks the gate spacers 116 and the top masks 114 of the dummy gate structures 106. For example, recessing the fins 104 may be performed by a dry chemical etch with a plasma source and an etchant gas. The plasma source may be inductively coupled plasma (ICR) etch, transformer coupled plasma (TCP) etch, electron cyclotron resonance (ECR) etch, reactive ion etch (RIE), or the like and the etchant gas may be fluorine, chlorine, bromine, combinations thereof, or the like, which etches the protruding fins 104 at a faster etch rate than it etches the gate spacers 116 and the top masks 114 of the dummy gate structures 106. In some other embodiments, recessing the protruding fins 104 may be performed by a wet chemical etch which etches the fins 104 at a faster etch rate than it etches the gate spacers 116 and the top masks 114 of the dummy gate structures 106. In some other embodiments, recessing the protruding fins 104 may be performed by a combination of a dry chemical etch and a wet chemical etch.

Once recesses are created in the source/drain regions of the fin 104, source/drain epitaxial structures 122 are formed in the source/drain recesses in the fin 104 by using one or more epitaxy or epitaxial (epi) processes that provides one or more epitaxial materials on the protruding fins 104. During the epitaxial growth process, the gate spacers 116 limit the one or more epitaxial materials to source/drain regions in the fin 104. In some embodiments, the lattice constants of the source/drain epitaxial structures 122 are different from the lattice constant of the fins 104, so that the channel region in the fin 104 and between the source/drain epitaxial structures 122 can be strained or stressed by the source/drain epitaxial structures 122 to improve carrier mobility of the semiconductor device and enhance the device performance. The epitaxy processes include CVD deposition techniques (e.g., PECVD, vapor-phase epitaxy (VPE) and/or ultra-high vacuum CVD (UHV-CVD)), molecular beam epitaxy, and/or other suitable processes. The epitaxy process may use gaseous and/or liquid precursors, which interact with the composition of the fins 104.

In some embodiments, the source/drain epitaxial structures 122 may include Ge, Si, GaAs, AlGaAs, SiGe, GaAsP, SiP, or other suitable material. The source/drain epitaxial structures 122 may be in-situ doped during the epitaxial process by introducing doping species including p-type dopants, such as boron or BF₂, n-type dopants, such as phosphorus or arsenic, and/or other suitable dopants including combinations thereof. If the source/drain epitaxial structures 122 are not in-situ doped, an implantation process (i.e., a junction implant process) is performed to dope the source/drain epitaxial structures 122. In some exemplary embodiments, the source/drain epitaxial structures 122 in an n-type transistor include SiP, while those in a p-type include GeSnB and/or SiGeSnB. In embodiments with different device types, a mask, such as a photoresist, may be formed over n-type device regions, while exposing p-type device regions, and p-type epitaxial structures may be formed on the exposed fins 104 in the p-type device regions. The mask may then be removed. Subsequently, a mask, such as a photoresist, may be formed over the p-type device region while exposing the n-type device regions, and n-type epitaxial structures may be formed on the exposed fins 104 in the n-type device region. The mask may then be removed.

Once the source/drain epitaxial structures 122 are formed, an annealing process can be performed to activate the p-type dopants or n-type dopants in the source/drain epitaxial structures 122. The annealing process may be, for example, a rapid thermal anneal (RTA), a laser anneal, a millisecond thermal annealing (MSA) process or the like.

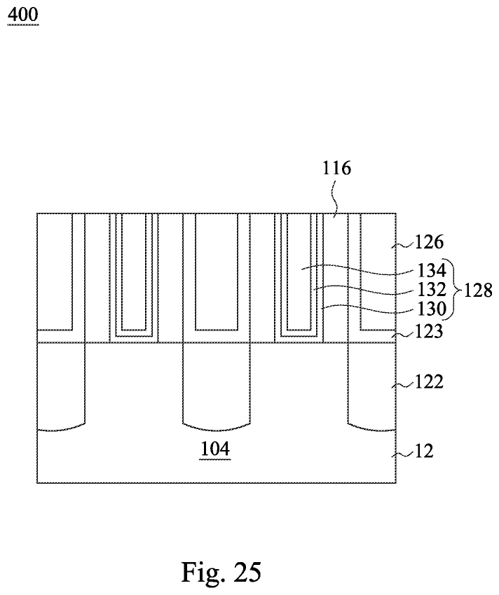

Next, in FIG. 25, a contact etch stop layer (CESL) 123 and an interlayer dielectric (ILD) layer 126 are formed on the substrate 12 in sequence. In some examples, the CESL 123 includes a silicon nitride layer, silicon oxide layer, a silicon oxynitride layer, and/or other suitable materials having a different etch selectivity than the ILD layer 126. The CESL 123 may be formed by plasma-enhanced chemical vapor deposition (PECVD) process and/or other suitable deposition or oxidation processes. In some embodiments, the ILD layer 126 includes materials such as tetraethylorthosilicate (TEOS) oxide, un-doped silicate glass, or doped silicon oxide such as borophosphosilicate glass (BPSG), fused silica glass (FSG), phosphosilicate glass (PSG), boron doped silicon glass (BSG), and/or other suitable dielectric materials having a different etch selectivity than the CESL 123. The ILD layer 126 may be deposited by a PECVD process or other suitable deposition technique. In some embodiments, after formation of the ILD layer 126, the wafer may be subject to a high thermal budget process to anneal the ILD layer 126.

In some examples, after forming the ILD layer 126, a planarization process may be performed to remove excessive materials of the ILD layer 126 and the CESL 123. For example, a planarization process includes a chemical mechanical planarization (CMP) process which removes portions of the ILD layer 126 and the CESL 123 overlying the dummy gate structures 106. In some embodiments, the CMP process also removes bottom masks 112 and top masks 114 (as shown in FIG. 25) and exposes the dummy gate electrodes 109.

An etching process is performed to remove the dummy gate electrode 109 and the dummy gate dielectric layer 108, resulting in gate trenches between corresponding gate spacers 116. The dummy gate structures 106 are removed using a selective etching process (e.g., selective dry etching, selective wet etching, or a combination thereof) that etches materials in the dummy gate structures 106 at a faster etch rate than it etches other materials (e.g., gate spacers 116 and/or the ILD layer 126).

Thereafter, replacement gate structures 128 are respectively formed in the gate trenches. The gate structures 128 may be the final gates of FinFETs. In FinFETs, the fins may be patterned by any suitable method. For example, the fins may be patterned using one or more photolithography processes, including double-patterning or multi-patterning processes. Generally, double-patterning or multi-patterning processes combine photolithography and self-aligned processes, allowing patterns to be created that have, for example, pitches smaller than what is otherwise obtainable using a single, direct photolithography process. For example, in one embodiment, a sacrificial layer is formed over a substrate and patterned using a photolithography process. Spacers are formed alongside the patterned sacrificial layer using a self-aligned process. The sacrificial layer is then removed, and the remaining spacers may then be used to pattern the fins. The final gate structures each may be a high-k/metal gate (HKMG) stack, however other compositions are possible. In some embodiments, each of the gate structures 128 forms the gate associated with the three-sides of the channel region provided by the fin 104. Stated another way, each of the gate structures 128 wraps around the fin 104 on three sides. In various embodiments, the high-k/metal gate structure 128 includes a gate dielectric layer 130 lining the gate trench, a work function metal layer 132 formed over the gate dielectric layer 130, and a fill metal 134 formed over the work function metal layer 132 and filling a remainder of gate trenches. The gate dielectric layer 130 includes an interfacial layer (e.g., silicon oxide layer) and a high-k gate dielectric layer over the interfacial layer. High-k gate dielectrics, as used and described herein, include dielectric materials having a high dielectric constant, for example, greater than that of thermal silicon oxide (˜3.9). The work function metal layer 132 and/or the fill metal 134 used within high-k/metal gate structures 128 may include a metal, metal alloy, or metal silicide. Formation of the high-k/metal gate structures 128 may include multiple deposition processes to form various gate materials, one or more liner layers, and one or more CMP processes to remove excessive gate materials.

In some embodiments, the interfacial layer of the gate dielectric layer 130 may include a dielectric material such as silicon oxide (SiO₂), HfSiO, or silicon oxynitride (SiON). The interfacial layer may be formed by chemical oxidation, thermal oxidation, atomic layer deposition (ALD), chemical vapor deposition (CVD), and/or other suitable method. The high-k dielectric layer of the gate dielectric layer 130 may include hafnium oxide (HfO2). Alternatively, the gate dielectric layer 130 may include other high-k dielectrics, such as hafnium silicon oxide (HfSiO), hafnium silicon oxynitride (HfSiON), hafnium tantalum oxide (HfTaO), hafnium titanium oxide (HfTiO), hafnium zirconium oxide (HfZrO), lanthanum oxide (LaO), zirconium oxide (ZrO), titanium oxide (TiO), tantalum oxide (Ta₂O₅), yttrium oxide (Y₂O₃), strontium titanium oxide (SrTiO₃, STO), barium titanium oxide (BaTiO3, BTO), barium zirconium oxide (BaZrO), hafnium lanthanum oxide (HfLaO), lanthanum silicon oxide (LaSiO), aluminum silicon oxide (AlSiO), aluminum oxide (Al₂O₃), silicon nitride (Si₃N₄), oxynitrides (SiON), and combinations thereof.

The work function metal layer 132 may include work function metals to provide a suitable work function for the high-k/metal gate structures 128. For an n-type FinFET, the work function metal layer 132 may include one or more n-type work function metals (N-metal). The n-type work function metals may exemplarily include, but are not limited to, titanium aluminide (TiAl), titanium aluminium nitride (TiAlN), carbo-nitride tantalum (TaCN), hafnium (Hf), zirconium (Zr), titanium (Ti), tantalum (Ta), aluminum (Al), metal carbides (e.g., hafnium carbide (HfC), zirconium carbide (ZrC), titanium carbide (TiC), aluminum carbide (AlC)), aluminides, and/or other suitable materials. On the other hand, for a p-type FinFET, the work function metal layer 132 may include one or more p-type work function metals (P-metal). The p-type work function metals may exemplarily include, but are not limited to, titanium nitride (TiN), tungsten nitride (WN), tungsten (W), ruthenium (Ru), palladium (Pd), platinum (Pt), cobalt (Co), nickel (Ni), conductive metal oxides, and/or other suitable materials.

In some embodiments, the fill metal 134 may exemplarily include, but are not limited to, tungsten, aluminum, copper, nickel, cobalt, titanium, tantalum, titanium nitride, tantalum nitride, nickel silicide, cobalt silicide, TaC, TaSiN, TaCN, TiAl, TiAlN, or other suitable materials.

In some embodiments, the semiconductor device 400 includes other layers or features not specifically illustrated. In some embodiments, back end of line (BEOL) processes are performed on the semiconductor device 400. In some embodiments, the semiconductor device 400 is formed by a non-replacement metal gate process or a gate-first process.

Based on the above discussions, it can be seen that the present disclosure offers advantages over conventional methods. It is understood, however, that other embodiments may offer additional advantages, and not all advantages are necessarily disclosed herein, and that no particular advantage is required for all embodiments. One advantage is that by including the high EUV absorbing group and a donor-acceptor design in the same molecule of the PAG, the high EUV absorbing group can absorb an EUV photon due to its inner-shell electrons and have a rapid intramolecular charge transfer from donor to acceptor. Another advantage is that a decomposition of the PAG can happen easily and quickly via intramolecular charge transfer. Yet another advantage is that since the PAG include both of the intermolecular charge transfer and the intramolecular charge transfer, an acid generation efficiency is improved, which is beneficial for improving lithography performance.

In some embodiments, a photo acid generator comprises a photoactive cation and an anion attached to the photoactive cation. The photoactive cation comprises a moiety and one or more EUV absorbing atoms attached to the moiety. The moiety includes onium salts, selenium salts, phosphonium salts, iodonium salts, or sulfonium salts. In some embodiments, the one or more EUV absorbing atoms comprise I, Sn, Xe, Te, Sb, In, Cs, At, Bi, Po, or a combination thereof. In some embodiments, the moiety is an aromatic or a non-aromatic ring having a substituted carbon group or functionalized group including —H, —F, —CF₃, —I, —Br, —Cl, —NH₂, —COOH, —OH, —SH, —N₃, —S(═O)—, alkene, alkyne, imine, ether, ester, aldehyde, ketone, amide, sulfone, acetic acid, cyanide, allene, alcohol, amine, phosphine, phosphite, aniline, pyridine, or pyrrole. In some embodiments, the photo acid generator has formula (A1) to (A12):

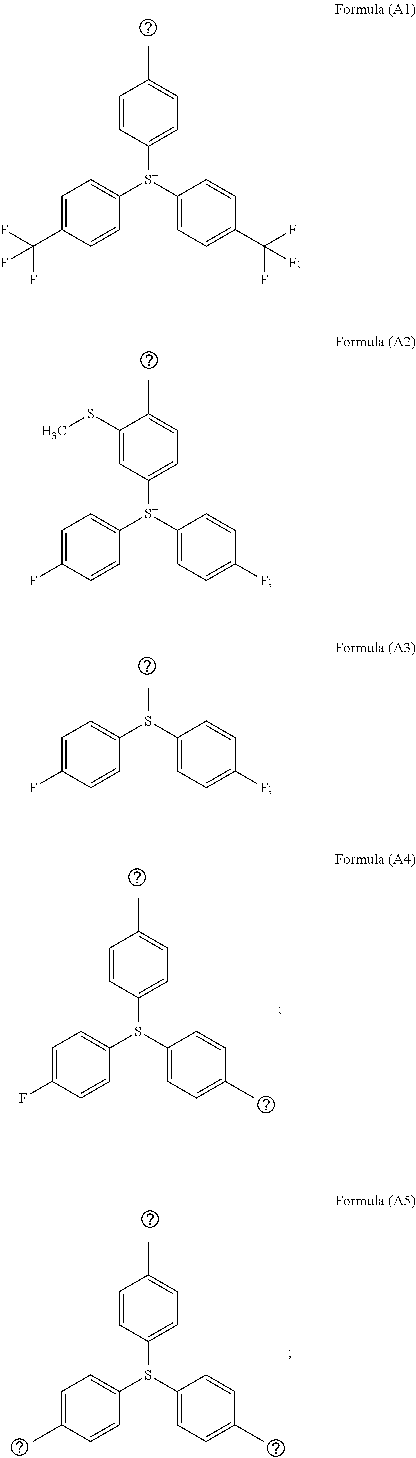

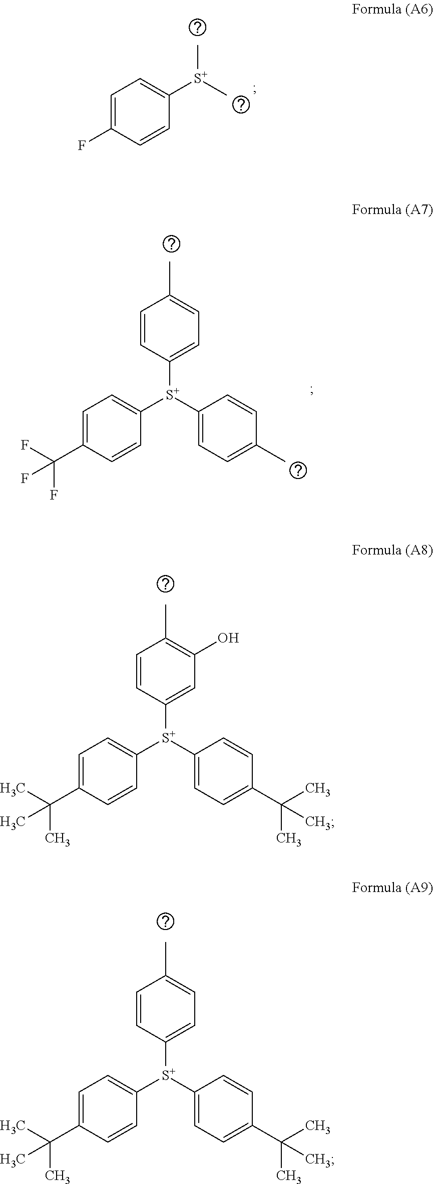

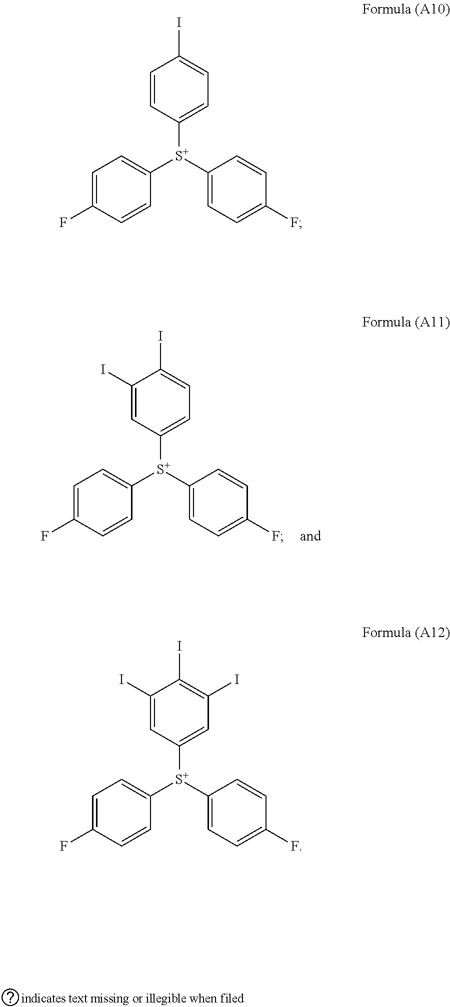 [SMILES: Fc1ccc([S+](c2ccc(F)cc2)c2ccc(I)cc2)cc1 ; Fc1ccc([S+](c2ccc(F)cc2)c2ccc(I)c(I)c2)cc1 ; Fc1ccc([S+](c2ccc(F)cc2)c2cc(I)c(I)c(I)c2)cc1]

In some embodiments, the one or more EUV absorbing atoms are UV absorbable. In some embodiments, the one or more EUV absorbing atoms are an electron donating group, and the moiety is an electron withdrawing group.

In some embodiments, a photoresist composition comprises a polymer, a photo acid generator and a solvent. The photo acid generator comprises a cation. The cation is made of one or more electron donor materials and an electron acceptor material attached to the one or more electron donor materials. The one or more electron donor materials are radiation absorbable. In some embodiments, the one or more electron donor materials comprise I, Sn, Xe, Te, Sb, In, Cs, At, Bi, Po, or a combination thereof. In some embodiments, the one or more electron donor materials are UV absorbable, EUV absorbable, or UV absorbable and EUV absorbable. In some embodiments, the cation of the photo acid generator has formulae (A1) to (A12):

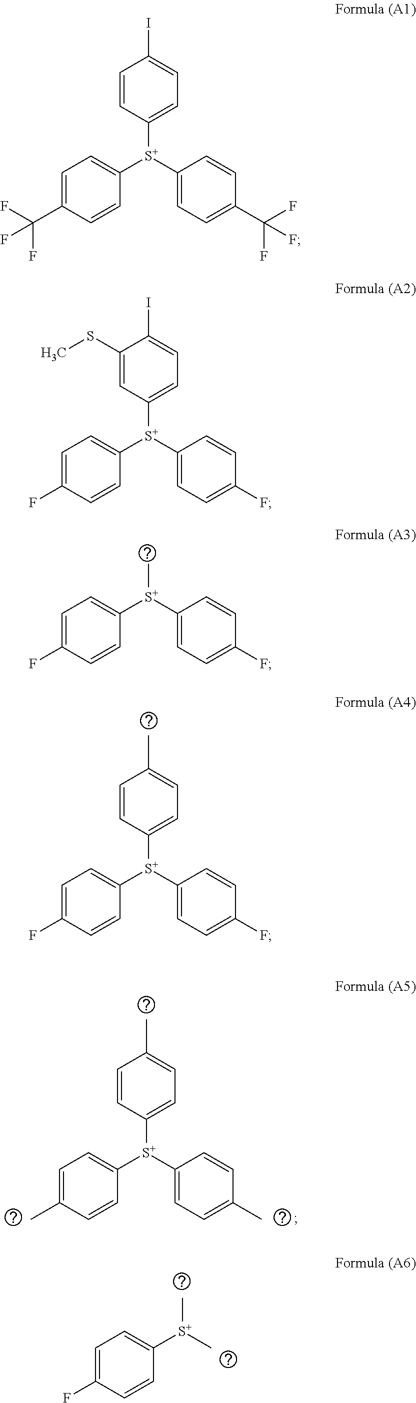

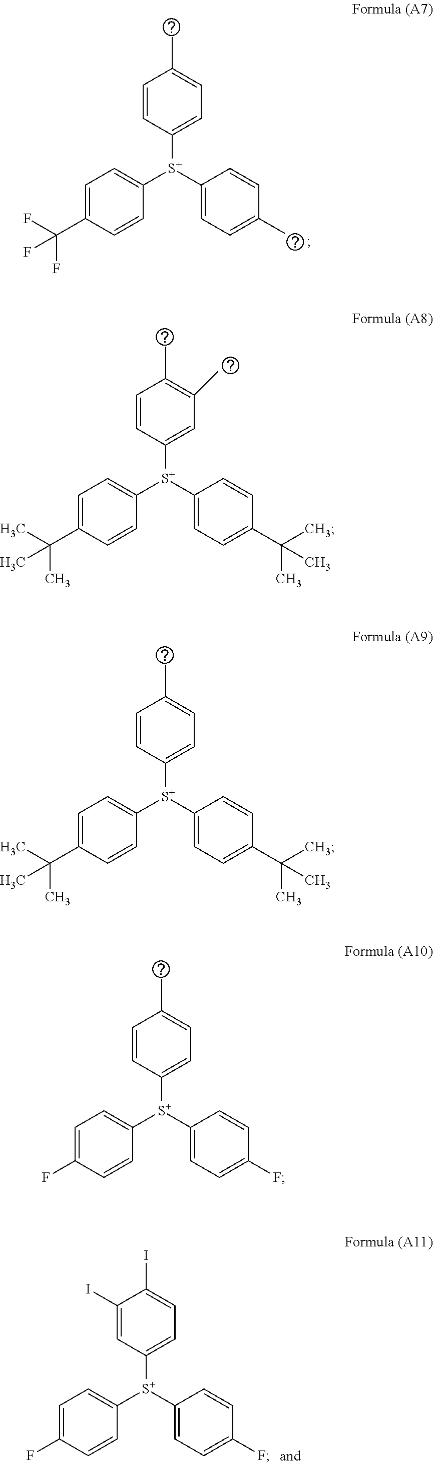

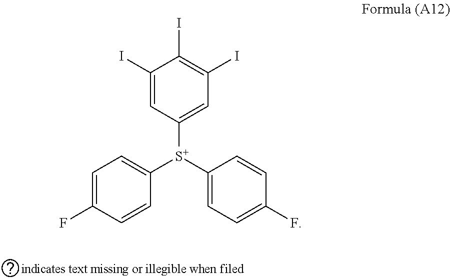 [SMILES: Fc1ccc([S+](c2ccc(F)cc2)c2cc(I)c(I)c(I)c2)cc1]

In some embodiments, the one or more electron donor materials have at least one 4d orbital electron. In some embodiments, the one or more electron donor materials have an EUV sensitive molecular orbital being 4d orbital. In some embodiments, the electron acceptor material of the cation comprises onium salts, selenium salts, phosphonium salts, iodonium salts or sulfonium salts. In some embodiments, the electron acceptor material of the cation is an aromatic or a non-aromatic ring having a substituted carbon group or functionalized group including —H, —F, —CF₃, —I, —Br, —Cl, —NH₂, —COOH, —OH, —SH, —N₃, —S(═O)—, alkene, alkyne, imine, ether, ester, aldehyde, ketone, amide, sulfone, acetic acid, cyanide, allene, alcohol, amine, phosphine, phosphite, aniline, pyridine, or pyrrole.

In some embodiments, a lithography method comprises the following steps. A photoresist composition is deposited on a substrate to form a photoresist layer, in which the photoresist composition comprises a polymer, a photo acid generator and a solvent. The photoresist layer is exposed such that the photo acid generator decomposes via charge transfer mechanisms with different types, and the charge transfer mechanisms comprise an intramolecular charge transfer. The photoresist layer is developed. The substrate is etched using the developed photoresist layer as an etch mask. In some embodiments, the charge transfer mechanisms further comprises an intermolecular charge transfer. In some embodiments, the photo acid generator comprises a cation and an anion attached to the cation. The cation comprises at least one atom having an EUV cross section being higher than about 1×10⁷ cm²/mol. In some embodiments, the at least one atom is I, Sn, Xe, Te, Sb, In, Cs, At, Bi, Po, or a combination thereof. In some embodiments, the at least one atom is an electron donating group. In some embodiments, the cation further comprises an electron withdrawing group attached to the at least one atom.

The foregoing outlines feature of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A photo acid generator, comprising:
a photoactive cation, wherein the photoactive cation comprises:
a moiety including onium salts, selenium salts, phosphonium salts, iodonium salts, or sulfonium salts; and
one or more EUV absorbing atoms attached to the moiety; and
an anion attached to the photoactive cation.

2. The photo acid generator of claim 1, wherein the one or more EUV absorbing atoms comprise I, Sn, Xe, Te, Sb, In, Cs, At, Bi, Po, or a combination thereof.

3. The photo acid generator of claim 1, wherein the moiety is an aromatic or a non-aromatic ring having a substituted carbon group or functionalized group including —H, —F, —CF₃, —I, —Br, —Cl, —NH₂, —COOH, —OH, —SH, —N₃, —S(═O)—, alkene, alkyne, imine, ether, ester, aldehyde, ketone, amide, sulfone, acetic acid, cyanide, allene, alcohol, amine, phosphine, phosphite, aniline, pyridine, or pyrrole.

4. The photo acid generator of claim 1, wherein the photo acid generator has formula (A1) to (A12):

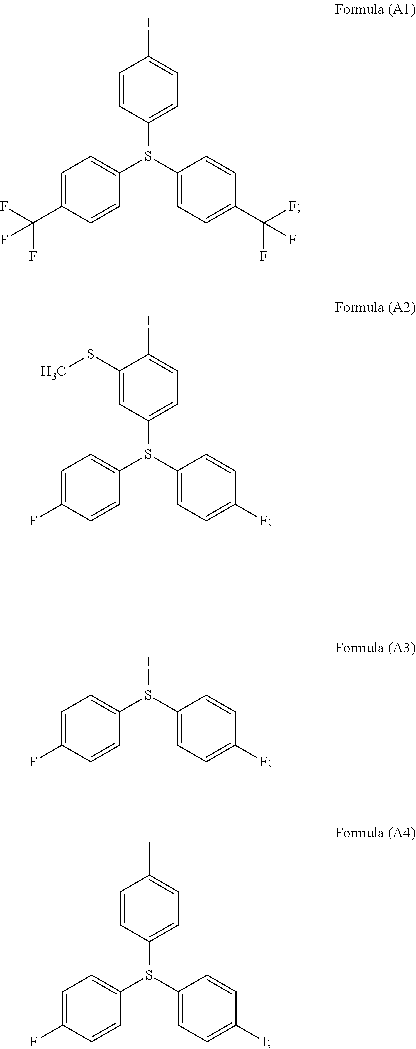 [SMILES: FC(F)(F)c1ccc([S+](c2ccc(I)cc2)c2ccc(C(F)(F)F)cc2)cc1 ; CSc1cc([S+](c2ccc(F)cc2)c2ccc(F)cc2)ccc1I ; Fc1ccc([S+](I)c2ccc(F)cc2)cc1 ; Cc1ccc([S+](c2ccc(F)cc2)c2ccc(I)cc2)cc1]

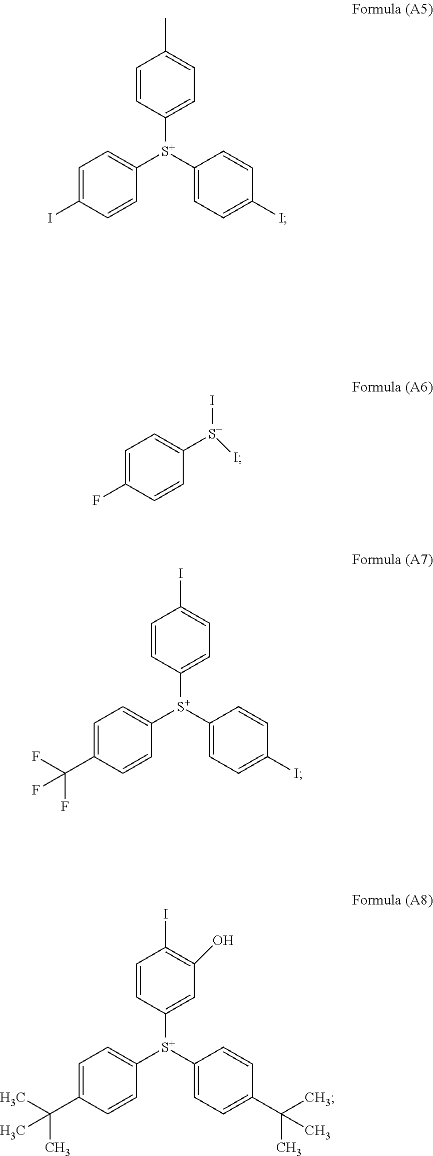 [SMILES: Cc1ccc([S+](c2ccc(I)cc2)c2ccc(I)cc2)cc1 ; Fc1ccc([S+](I)I)cc1 ; FC(F)(F)c1ccc([S+](c2ccc(I)cc2)c2ccc(I)cc2)cc1 ; CC(C)(C)c1ccc([S+](c2ccc(C(C)(C)C)cc2)c2ccc(I)c(O)c2)cc1]

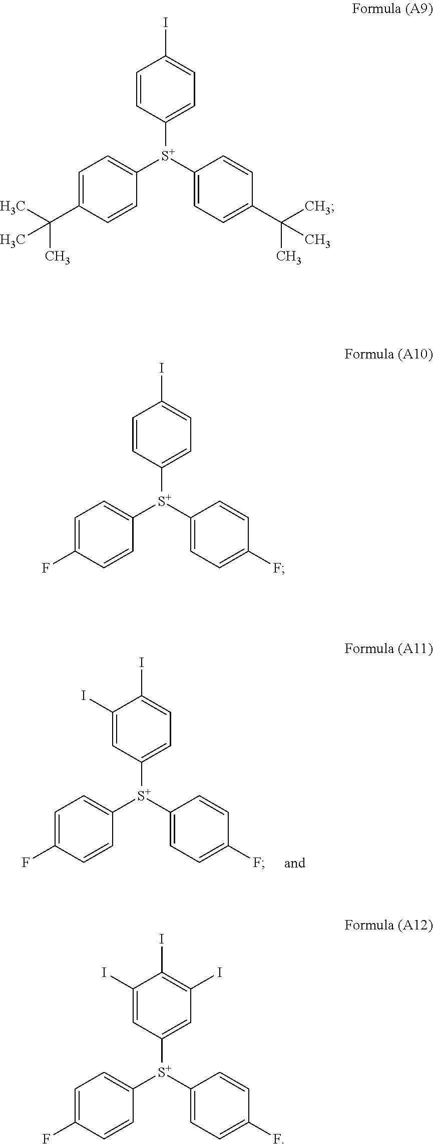 [SMILES: CC(C)(C)c1ccc([S+](c2ccc(I)cc2)c2ccc(C(C)(C)C)cc2)cc1 ; Fc1ccc([S+](c2ccc(F)cc2)c2ccc(I)cc2)cc1 ; Fc1ccc([S+](c2ccc(F)cc2)c2ccc(I)c(I)c2)cc1 ; Fc1ccc([S+](c2ccc(F)cc2)c2cc(I)c(I)c(I)c2)cc1]

5. The photo acid generator of claim 1, wherein the one or more EUV absorbing atoms are UV absorbable.

6. The photo acid generator of claim 1, wherein the one or more EUV absorbing atoms are an electron donating group, and the moiety is an electron withdrawing group.

7. A photoresist composition, comprising:
a polymer;
a photo acid generator, wherein the photo acid generator comprises a cation, and the cation is made of:
one or more electron donor materials, the one or more electron donor materials being radiation absorbable; and
an electron acceptor material attached to the one or more electron donor materials; and
a solvent.

8. The photoresist composition of claim 7, wherein the one or more electron donor materials comprise I, Sn, Xe, Te, Sb, In, Cs, At, Bi, Po, or a combination thereof.

9. The photoresist composition of claim 7, wherein the one or more electron donor materials are UV absorbable, EUV absorbable, or UV absorbable and EUV absorbable.

10. The photoresist composition of claim 7, wherein the cation of the photo acid generator has formulae (A1) to (A12):

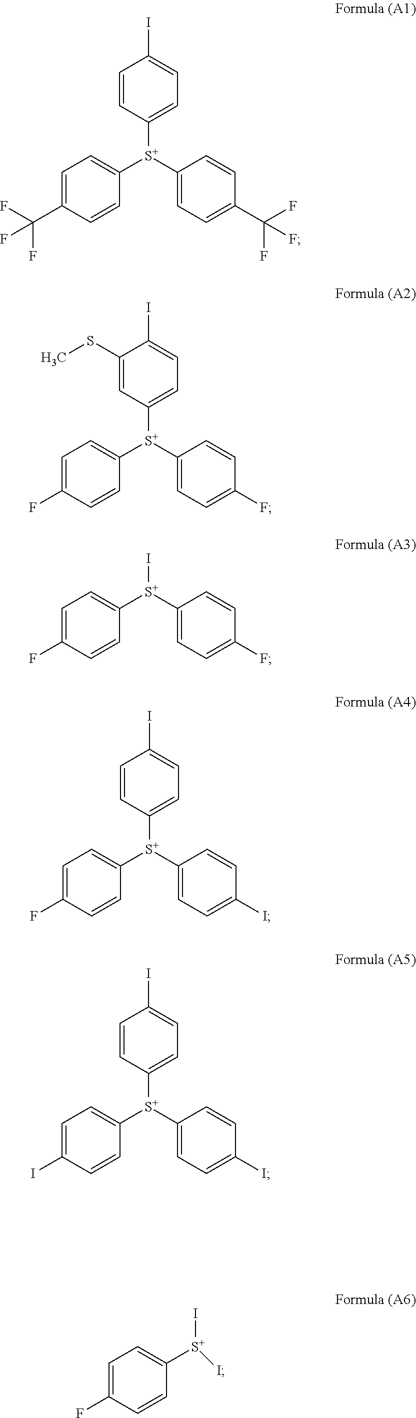 [SMILES: FC(F)(F)c1ccc([S+](c2ccc(I)cc2)c2ccc(C(F)(F)F)cc2)cc1 ; CSc1cc([S+](c2ccc(F)cc2)c2ccc(F)cc2)ccc1I ; Fc1ccc([S+](I)c2ccc(F)cc2)cc1 ; Fc1ccc([S+](c2ccc(I)cc2)c2ccc(I)cc2)cc1 ; Ic1ccc([S+](c2ccc(I)cc2)c2ccc(I)cc2)cc1 ; Fc1ccc([S+](I)I)cc1]

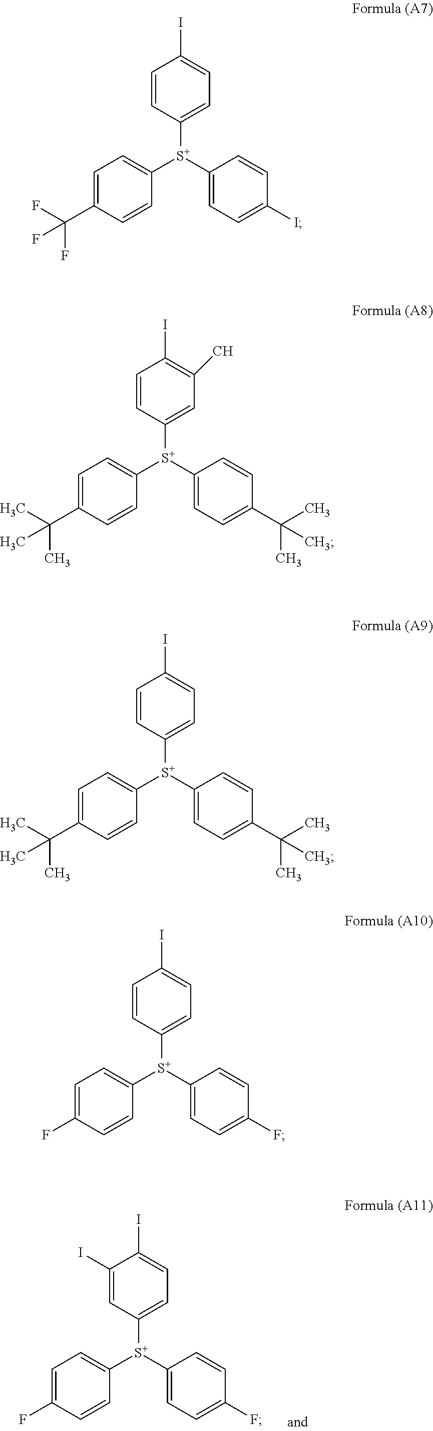 [SMILES: FC(F)(F)c1ccc([S+](c2ccc(I)cc2)c2ccc(I)cc2)cc1 ; Cc1cc([S+](c2ccc(C(C)(C)C)cc2)c2ccc(C(C)(C)C)cc2)ccc1I ; CC(C)(C)c1ccc([S+](c2ccc(I)cc2)c2ccc(C(C)(C)C)cc2)cc1 ; Fc1ccc([S+](c2ccc(F)cc2)c2ccc(I)cc2)cc1 ; Fc1ccc([S+](c2ccc(F)cc2)c2ccc(I)c(I)c2)cc1]

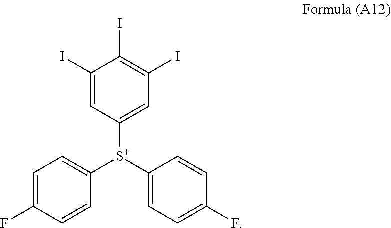 [SMILES: Fc1ccc([S+](c2ccc(F)cc2)c2cc(I)c(I)c(I)c2)cc1]

11. The photoresist composition of claim 7, wherein the one or more electron donor materials have at least one 4d orbital electron.

12. The photoresist composition of claim 7, wherein the one or more electron donor materials have an EUV sensitive molecular orbital being 4d orbital.

13. The photoresist composition of claim 7, wherein the electron acceptor material of the cation comprises onium salts, selenium salts, phosphonium salts, iodonium salts or sulfonium salts.

14. The photoresist composition of claim 7, wherein the electron acceptor material of the cation is an aromatic or a non-aromatic ring having a substituted carbon group or functionalized group including —H, —F, —CF₃, —I, —Br, —Cl, —NH₂, —COOH, —OH, —SH, —N₃, —S(═O)—, alkene, alkyne, imine, ether, ester, aldehyde, ketone, amide, sulfone, acetic acid, cyanide, allene, alcohol, amine, phosphine, phosphite, aniline, pyridine, or pyrrole.

15. A lithography method, comprising:
depositing a photoresist composition on a substrate to form a photoresist layer, wherein the photoresist composition comprises:
a polymer;
a photo acid generator; and
a solvent;
exposing the photoresist layer such that the photo acid generator decomposes via charge transfer mechanisms with different types, and the charge transfer mechanisms comprise an intramolecular charge transfer;
developing the photoresist layer; and
etching the substrate using the developed photoresist layer as an etch mask.

16. The method of claim 15, wherein the charge transfer mechanisms further comprises an intermolecular charge transfer.

17. The method of claim 15, wherein the photo acid generator comprises:
a cation, wherein the cation comprises at least one atom having an EUV cross section being higher than about 1×10⁷ cm²/mol; and
an anion attached to the cation.

18. The method of claim 17, wherein the at least one atom is I, Sn, Xe, Te, Sb, In, Cs, At, Bi, Po, or a combination thereof.

19. The method of claim 17, wherein the at least one atom is an electron donating group.

20. The method of claim 17, wherein the cation further comprises:
an electron withdrawing group attached to the at least one atom.
